(19) United States
(12) Patent Application Publication
(10) Pub. No.: US 2026/0159509 A1
(43) Pub. Date: Jun. 11, 2026

(54) EXTRACTANT COMPOUNDS AND THEIR USE FOR THE SEPARATION OF RARE EARTH ELEMENTS

(71) Applicant: UT-Battelle, LLC, Oak Ridge, TN (US)

(72) Inventors: Nikki THIELE, Kingston, TN (US); Benjamin L. DOUGHTY, Knoxville, TN (US); Md Faizul ISLAM, Knoxville, TN (US); Lu LIN, Philadelphia, PA (US)

(21) Appl. No.: 19/414,446

(22) Filed: Dec. 10, 2025

Related U.S. Application Data
(60) Provisional application No. 63/730,473, filed on Dec. 11, 2024.

(51) Int. Cl. C07D 413/12; C22B 3/36; C22B 59/00

(52) U.S. Cl. CPC C07D 413/12; C22B 3/36; C22B 59/00

(57) ABSTRACT

A metal chelator compound having the following structure:

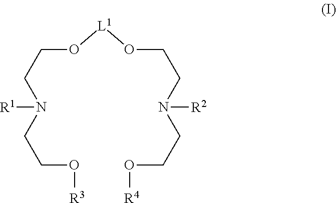

wherein: L¹ is a linker containing 2-8 carbon atoms; R¹ and R² are independently selected from hydrocarbon groups containing 1-12 carbon atoms, and each one of R¹ and R² is substituted with at least one group selected from: carboxy groups —C(O) OR′; phosphoryl groups —P(═O)(OH)R″; oxy groups OR′; amino groups —NR′₂; and amido groups —C(O)NHR′₂; wherein R′ is independently selected from H and hydrocarbon groups containing 1-6 carbon atoms and R″ is selected from OH and hydrocarbon groups containing 1-6 carbon atoms; and R³ and R⁴ are independently selected from hydrocarbon groups (optionally fluorinated) containing 4-30 carbon atoms. Also described herein is a hydrophobic solution containing one or more metal chelators under Formula (I), metal-ligand complexes containing a metal chelator under Formula (I), and a method of separating rare earth elements from an aqueous solution by use of the hydrophobic solution.

### CROSS REFERENCE TO RELATED APPLICATION

The present application claims benefit of U.S. Provisional Application No. 63/730,473, filed on Dec. 11, 2024, all of the contents of which are incorporated herein by reference.

### STATEMENT REGARDING FEDERALLY SPONSORED RESEARCH

This invention was made with government support under Prime Contract No. DE-AC05-00OR22725 awarded by the U.S. Department of Energy. The government has certain rights in the invention.

### FIELD OF THE INVENTION

The present invention generally relates to metal chelating compounds, particularly lanthanide-complexing ligands, and their use in separating rare earth and lanthanide elements. The present invention more particularly relates to macrocycle-mimicking complexing agents and methods of using them for facilitating the separation of rare earth and lanthanide elements.

### BACKGROUND

The demand for lanthanides (Ln), comprising the 15 elements La through Lu, has skyrocketed over the last several decades due to their utility in modern technology and medicine. They can be found, for example, in batteries and high-performance magnets used in clean energy and national security applications, in phosphors for lighted displays, and even in many radiopharmaceutical agents for diagnosis and treatment of cancer and other diseases. Leveraging the distinctive properties of Ln in these applications requires not only the ability to extract a sufficient supply from dilute, complex sources, but also a means to further separate them from each other. This separation, however, is notoriously challenging because their chemical properties in aqueous solution are very similar.

A commonly exploited characteristic used to separate Ln³⁺ ions is their larger-than-expected decrease in ionic radius with increasing atomic number, a trend known as the lanthanide contraction. This phenomenon renders the smaller, heavier Ln more densely charged than the larger, lighter Ln, consequently increasing the strength of their ionic bonding interactions with chelating ligands. Accordingly, smaller Ln generally form more stable coordination complexes than their larger congeners, driving conventional trends in selectivity for various separation schemes across the series. Although useful, this selectivity pattern for small over large Ln limits the development of separations targeting certain elements from mixtures that may be of higher value or exist in lower abundance. As such, new chelators that exhibit unconventional types of selectivity are needed.

### SUMMARY

In a first aspect, the present disclosure is directed to macrocycle-mimicking yet acyclic metal chelator compounds that exhibit a special characteristic of more strongly binding with larger radii or lower atomic number rare earth (RE) elements compared to smaller radii or higher atomic number RE elements. This novel characteristic permits an alternative means for separating RE elements than the conventional means, which can then permit separations of elements from atypical mixtures that may be of higher value or exist in lower abundance than conventionally practiced.

The metal chelator compounds that exhibit these novel properties have the following structure:

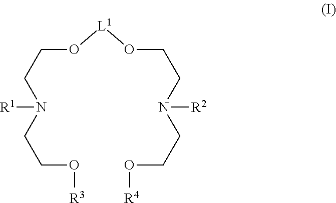

wherein: L¹ is a linker containing 2-8 carbon atoms optionally containing one or more heteroatoms selected from O, N, and S atoms; R¹ and R² are independently selected from hydrocarbon groups containing 1-12 carbon atoms optionally containing one or more heteroatoms selected from O, N, and S atoms, and each one of R¹ and R² is substituted with (i.e. contains) at least one heteroatom-containing group selected from: carboxy groups of the formula —C(O) OR′; phosphoryl groups of the formula —P(═O)(OH)R″; oxy groups of the formula OR′; amino groups of the formula —NR′2; and amido groups of the formula —C(O)NHR′2; wherein R′ is independently selected from H and hydrocarbon groups containing 1-6 carbon atoms and R″ is selected from OH and hydrocarbon groups containing 1-6 carbon atoms; and R³ and R⁴ are independently selected from hydrocarbon groups containing 4-30 carbon atoms.

In particular embodiments, L¹ is a linking group selected from one of an ethyl, a propyl, a diethyl ether, a cyclohexyl, and a benzyl; wherein each of R¹ and R² is a moiety including a terminal group independently selected from one of a carboxylic acid, a phosphinic acid, a phosphonic acid, a phenol, an amide, a carboxylic acid ester, a phosphinic acid ester, a phosphonic acid ester, and a phenol ether; and wherein R³ and R⁴ are each selected from one of a linear or distally branched alkyl group containing 1-30 carbon atoms.

More particularly, L¹ may be selected from any of the foregoing specific linkers:

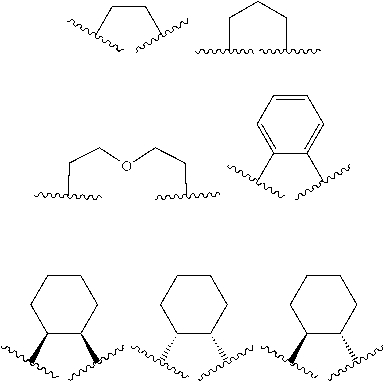

In some embodiments, one or both of R¹ and R² is or includes a picolinic acid group. In separate or further embodiments, R³ and R⁴ are each a linear alkyl or branched alkyl. In separate or further embodiments, (i) R¹ and R² are the same; (ii) R³ and R⁴ are the same; or (iii) both (i) and (ii).

In some embodiments, the compound of Formula (I) has any one of the following formulas:

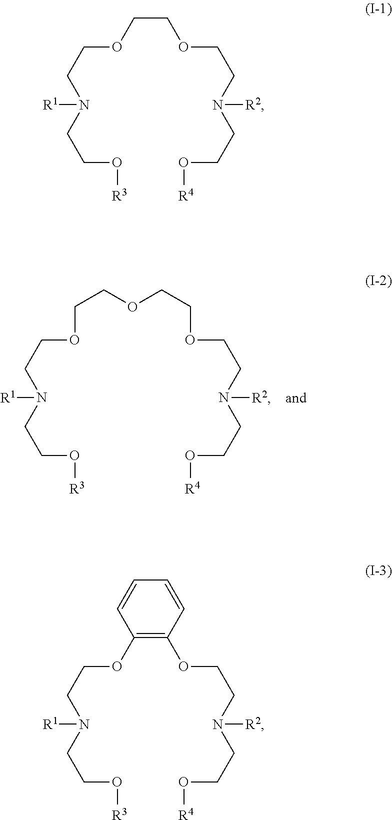

wherein R¹, R², R³, and R⁴ are as defined above under Formula (I). Any selections and combinations of R¹, R², R³, and R⁴, as provided in this disclosure, can be made in Formulas (I-1), (I-2), and (I-3).

In separate or further embodiments for Formula (I) or any sub-formula thereof, R¹ and R² may be independently selected from:

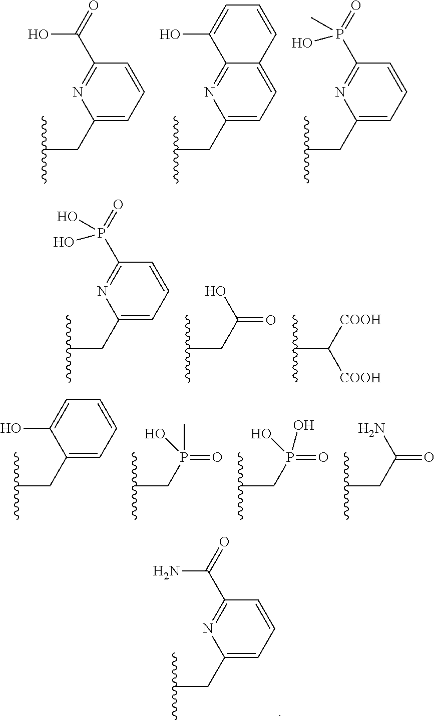

Any of the above species of R¹ and/or R² may be combined with any of the species provided above for L¹ in Formula (1) or any sub-formula thereof. Moreover, any selection of R³ and R⁴, as provided in this disclosure, can be made and combined with any of the above species of R¹ and/or R² may be combined with any of the species provided above for L¹ in Formula (1).

In separate or further embodiments of Formula (I) or any sub-formula thereof, R³ and R⁴ may be independently selected from: hydrocarbon groups (or more particularly, linear or branched alkyl groups) containing 5-30 carbon atoms, hydrocarbon groups (or more particularly, linear or branched alkyl groups) containing 6-30 carbon atoms, hydrocarbon groups (or more particularly, linear or branched alkyl groups) containing 7-30 carbon atoms, or hydrocarbon groups (or more particularly, linear or branched alkyl groups) containing 8-30 carbon atoms. Any of the above selections for R³ and R⁴ may be combined with any of the above species of R¹ and/or R² and these may be combined with any of the species provided above for L¹ in Formula (1) or any sub-formula thereof.

In some embodiments, the compound of Formula (I) has the following formula:

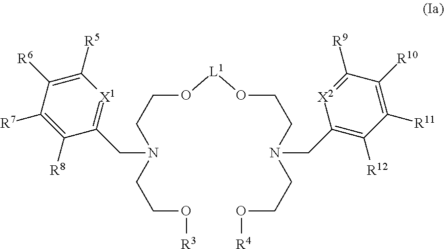

wherein L¹, R³, and R⁴ are as defined anywhere above; R⁵, R⁶, R⁷, R⁸, R⁹, R¹⁰, R¹¹, and R¹² are independently selected from H, hydrocarbon groups containing 1-3 carbon atoms; carboxy groups of the formula —C(O) OR′; phosphoryl groups of the formula —P(═O)(OH)R″; oxy groups of the formula OR′; amino groups of the formula —NR′2; and amido groups of the formula —C(O)NHR′2; wherein R′ is independently selected from H and hydrocarbon groups containing 1-6 carbon atoms and R″ is selected from OH and hydrocarbon groups containing 1-6 carbon atoms; wherein a pair of adjacent groups selected from R⁵, R⁶, R⁷, and R⁸ and/or a pair of adjacent groups selected from R⁹, R¹⁰, R¹¹, and R¹² are optionally interconnected to form an additional ring to form a fused ring system, wherein the additional ring may optionally and independently be substituted with one or more hydrocarbon groups containing 1-3 carbon atoms; carboxy groups of the formula —C(O) OR′; phosphoryl groups of the formula —P(═O)(OH)R″; oxy groups of the formula OR′; amino groups of the formula —NR′₂; and amido groups of the formula —C(O)NHR′₂; wherein R′ is independently selected from H and hydrocarbon groups containing 1-6 carbon atoms and R″ is selected from OH and hydrocarbon groups containing 1-6 carbon atoms; and X¹ is selected from CR^{a} and N, wherein R^{a} is selected from hydrocarbon groups containing 1-3 linearly connected carbon atoms; carboxy groups of the formula —C(O) OR′; phosphoryl groups of the formula —P(═O)(OH)R″; oxy groups of the formula OR′; amino groups of the formula —NR′₂; and amido groups of the formula —C(O)NHR′₂; wherein R′ is independently selected from H and hydrocarbon groups containing 1-6 carbon atoms and R″ is selected from OH and hydrocarbon groups containing 1-6 carbon atoms; X² is selected from CR^{b} and N, wherein R^{b} is selected from hydrocarbon groups containing 1-3 linearly connected carbon atoms; carboxy groups of the formula —C(O) OR′; phosphoryl groups of the formula —P(═O)(OH)R″; oxy groups of the formula OR′; amino groups of the formula —NR′₂; and amido groups of the formula —C(O)NHR′₂; wherein R′ is independently selected from H and hydrocarbon groups containing 1-6 carbon atoms and R″ is selected from OH and hydrocarbon groups containing 1-6 carbon atoms; wherein at least one of R^{a}, R⁵, R⁶, R⁷, and R⁸ is selected from the group consisting of said carboxy, phosphoryl, oxy, amino, and amido groups, and at least one of R^{b}, R⁹, R¹⁰, R¹¹, and R¹² is selected from the group consisting of said carboxy, phosphoryl, oxy, amino, and amido groups. In some embodiments, R⁵, R⁶, R⁷, R⁸, R⁹, R 10, R¹¹, and R¹² are all H.

In some embodiments, the compound of Formula (Ia) has any one of the following chemical structures:

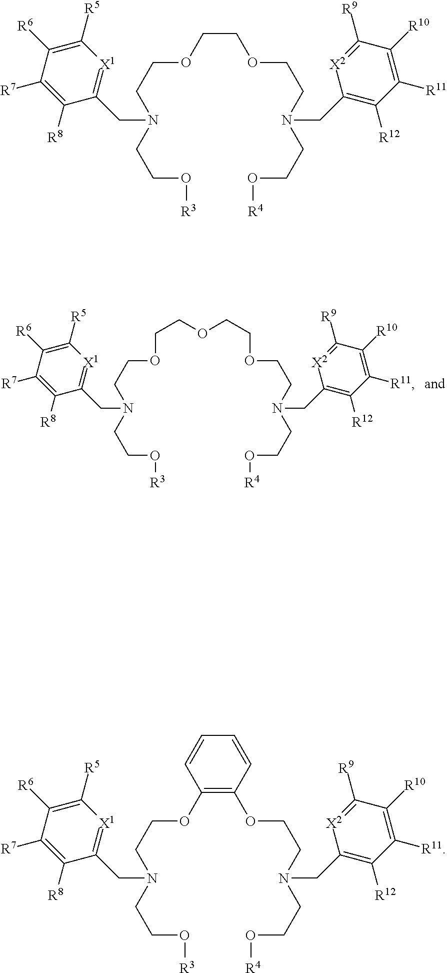

wherein any of R³, R⁴, R⁵—R¹², X¹, and X² variables in any of the above formulas may be independently selected from any of the definitions or exemplifications provided above and combined to arrive at one or more specific or sub-generic species. In some embodiments, R⁵—R¹² are all H.

In some embodiments, the compound of Formula (I) has the following formula:

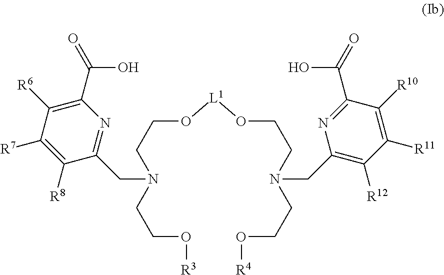

wherein L¹, R³, R⁴, R⁶—R⁸, and R¹⁰—R¹² are as defined anywhere above and may be independently selected from any of the definitions or exemplifications provided above and combined to arrive at one or more specific or sub-generic species. In some embodiments, R⁶—R⁸ and R¹⁰—R¹² are all H.

In some embodiments, the compound of Formula (Ib) has any one of the following chemical structures:

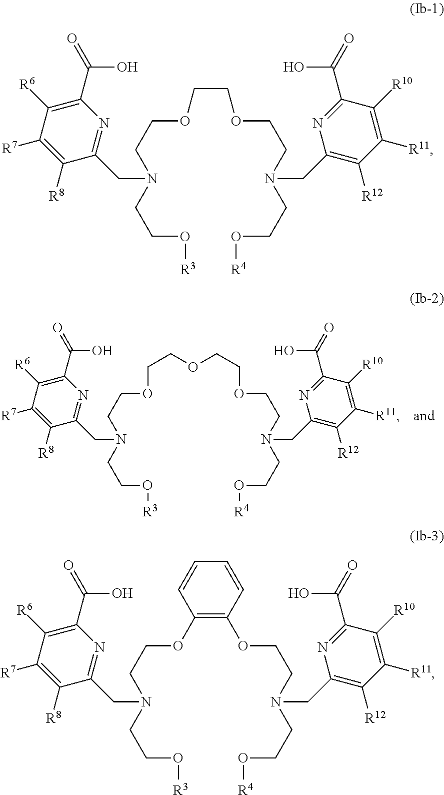

wherein any of R³, R⁴, R⁶—R⁸, and R¹⁰—R¹² variables in any of the above formulas may be independently selected from any of the definitions or exemplifications provided above and combined to arrive at one or more specific or sub-generic species. In some embodiments, R⁶—R⁸ and R¹⁰—R¹² are all H.

In some embodiments, the extractant compound has the following specific chemical structure:

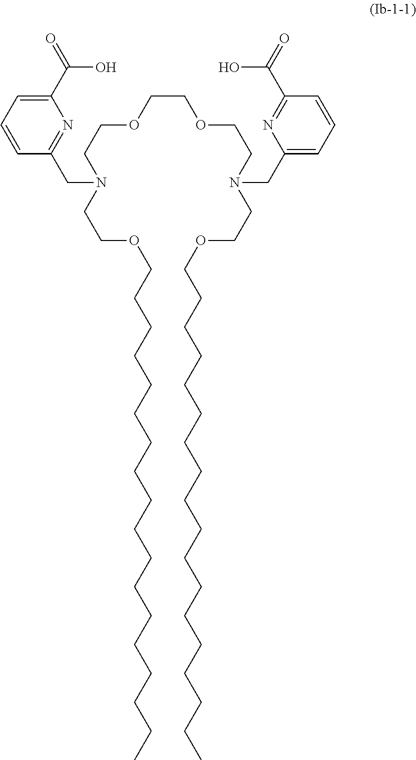 [SMILES: CCCCCCCCCCCCCCCCCCOCCN(CCOCCOCCN(CCOCCCCCCCCCCCCCCCCCC)Cc1cccc(C(=O)O)n1)Cc1cccc(C(=O)O)n1]

In some embodiments, the extractant compound has the following specific chemical structure:

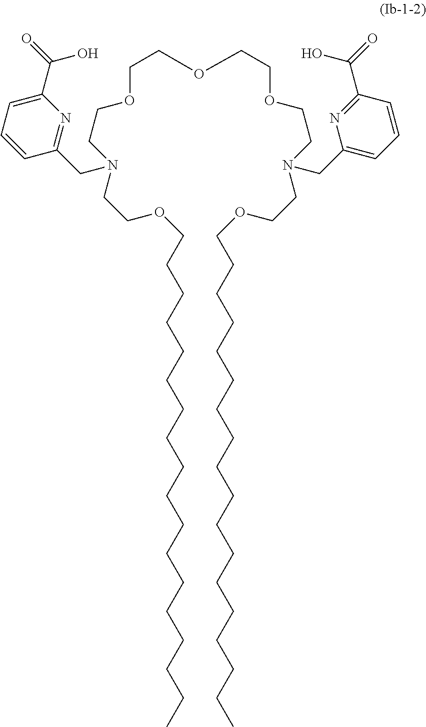 [SMILES: CCCCCCCCCCCCCCCCCCOCCN(CCOCCOCCOCCN(CCOCCCCCCCCCCCCCCCCCC)Cc1cccc(C(=O)O)n1)Cc1cccc(C(=O)O)n1]

In some embodiments, the extractant compound has the following specific chemical structure:

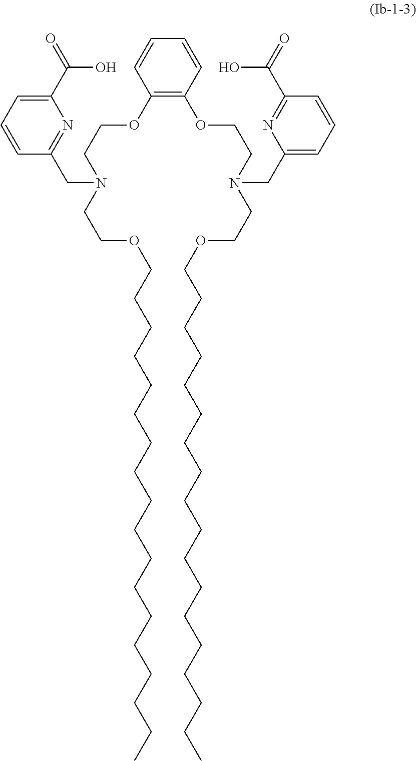 [SMILES: CCCCCCCCCCCCCCCCCCOCCN(CCOc1ccccc1OCCN(CCOCCCCCCCCCCCCCCCCCC)Cc1cccc(C(=O)O)n1)Cc1cccc(C(=O)O)n1]

In another aspect, the present disclosure is directed to an aqueous-insoluble hydrophobic solution useful for extracting rare earth elements from aqueous solutions, wherein the aqueous-insoluble hydrophobic solution contains a rare earth extractant compound (i.e., “extractant compound”) dissolved in an aqueous-insoluble hydrophobic solvent. The rare earth extractant compound may be any of the above-described metal chelator compounds according to Formula (1) or any sub-formula or specific species therein. In some embodiments, the aqueous-insoluble hydrophobic solvent is selected from a hydrocarbon solvent (halogenated or non-halogenated), alcohol solvent containing at least six carbon atoms, or an ether solvent, or a combination of any of these.

In another aspect, the present disclosure is directed to a metal-ligand complex containing the following components: (i) a metal chelator compound which may be any of the above-described metal chelator compounds according to Formula (1) or any sub-formula or specific species therein; and (ii) a metal (M) complexed with the metal chelator compound. In some embodiments, M is a rare earth metal, such as any one of lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), yttrium (Y), and scandium (Sc). In other embodiments, the M is a transition metal. In other embodiments, Mis an actinide metal.

In another aspect, the present disclosure is directed to a method of at least partially separating rare earth (RE) elements, such as any two or more from those disclosed above, from an aqueous solution containing at least two rare earth elements. More particularly, the method includes contacting the aqueous solution with an aqueous-insoluble (aqueous-immiscible) hydrophobic solution, as described above, containing a rare earth extractant compound dissolved in an aqueous-insoluble hydrophobic solvent. The rare earth extractant compound may be any of the above-described metal chelator compounds according to Formula (1) or any sub-formula or specific species therein. After contact, the aqueous-insoluble hydrophobic solution contains a higher molar ratio of lower atomic number or larger radii RE element(s) over higher atomic number or smaller radii RE element(s) compared to the molar ratio of lower atomic number or larger radii RE element(s) over higher atomic number or smaller radii RE element(s) in the aqueous solution. In some embodiments, the aqueous solution further includes a buffer that maintains the aqueous solution in a pH in a range of 2-7, 3-7, 2-6, or 3-6, or more particularly a lactate buffer. In separate or further embodiments, the aqueous solution further includes an electrolyte, such as NaCl and/or KCl. In some embodiments, the aqueous-immiscible solvent is selected from any of those exemplified above, or more particularly, a halogenated solvent, a hydrocarbon solvent, an aliphatic alcohol solvent, or mixtures thereof. In one embodiment, the aqueous-immiscible solvent is or includes 1,1,2,2-tetrachloroethane.

In stark contrast to the conformationally rigid ligands of the conventional paradigm, conformationally flexible ones are generally considered incapable of showing reverse (or any unconventional) Ln selectivity. Taking cues from nature and the concept of induced-fit recognition, flexible ligands may contort themselves to accommodate a variety of ionic radii such that the charge density of the heavier Ln, which drives the formation of stronger bonds, should dominate selectivity trends. However, biological systems leverage conformational flexibility to enhance selectivity by efficiently dispersing excess energy away from binding sites via coupling to low energy vibrational modes in the outer sphere.

A particular aim of the present work is the design of flexible ligands capable of providing unconventional size selectivity in Ln separation schemes. As part of this effort, and as discussed further in the Examples section, the present disclosure is particularly directed to a flexible amphiphilic ligand, coined octadecyl acyclopa (ODA), that features a diaza polyethylene glycol podand appended with two chelating picolinic acid pendants and two octa-decyl endcap groups. The aforesaid molecule is derived from the cyclic macropa chelator, in which a single bond has been ‘cut’, thus unraveling the macrocycle into a linear analog. Extraction experiments show that despite the loss of conformational rigidity afforded by the macrocyclic backbone of macropa, the linear ODA extractant is capable of preferentially stabilizing lighter Ln³⁺ ions versus the heavier species, in contrast to the conventional view of lanthanide coordination chemistry. Surface-specific vibrational sum frequency generation (SFG) spectroscopy shows that ODA arranges at organic/aqueous interfaces to form pseudocyclic structures, analogous to macropa, that are transported to the organic phase on extraction. The interfacial structures are found to be independent of the specific Ln³⁺ ions, which indicates that the stabilization is realized thermodynamically vs. kinetically. This finding is supported by ultrafast two-dimensional infrared spectroscopy (2D IR), density functional theory (DFT) calculations, and ab initio molecular dynamics (AIMD) simulations that connect conformation-al dynamics, binding energies, and strain to selectivity.

These results show that the reverse-size selectivity arises from the inability of the flexible ligand to adopt the optimal geometry needed to effectively coordinate the heavier, smaller lanthanides, despite the ability to form individually stronger bonding interactions. It is also herein shown that for molecules of this size, energy dissipation to outer-sphere constituents plays a secondary role to the number and strength of inner-sphere coordination interactions. These results point to a new paradigm in ligand design where conformational flexibility is embraced with an eye toward engineering collective interactions in Ln³⁺ coordination.

### BRIEF DESCRIPTION OF THE DRAWINGS

FIGS. 1A-1B. FIG. 1A is a graph showing extraction of Ln³⁺ ions from pH 4 lactate buffer by ODA in TCE, plotted as the log of conditional extraction coefficients, Kex, versus Ln³⁺ ionic radius. Error bars are not shown because the uncertainty is smaller than the symbols in the plot. FIG. 1B is a graph showing separation factors of adjacent Ln³⁺ pairs obtained from ODA solvent extraction experiments.

FIGS. 2A-2B. Structural characterization of Ln-ODA complexes at the organic/aqueous interface. FIG. 2A shows SFG spectra collected in the SSP polarization combination at pH 4.0. Spectra are offset for clarity. FIG. 2B shows a sketch of the proposed metal-binding conformation of ODA at the oil/aqueous interface. A representative Ln³⁺ ion is denoted as a large sphere toward near the interface. The arrows represent the measured transition dipoles of CH₂ groups discussed in the text.

FIGS. 3A-3C. Spectroscopic characterization of ODA complexes extracted into the bulk organic phase. FIG. 3A shows FTIR spectra of the carboxylate region taken from different Ln-ODA complexes in TCE, as described in the text. FIG. 3B shows measured 2D IR spectra from Ln-ODA complexes at two different waiting times, indicated on each plot. FIG. 3C is a graph showing spectral diffusion dynamics extracted from the 2D IR data.

FIGS. 4A-4B. FIG. 4A shows DFT-optimized structures of the ODAMe complexes with La³⁺, Gd³⁺, and Lu³⁺ ions. Solid lines represent the stronger Ln-O or Ln-N bonds in ODAMe complexes, whereas dashed lines represent the weaker Ln-O and Ln-N bonds in the Gd— and Lu-ODAMe complexes. FIG. 4B shows variation of the Ln-ligand bond distances (Å) as a function of simulation time, analyzed from the AIMD trajectories for the complexes of ODAMe with La³⁺ and Lu³⁺.

### DETAILED DESCRIPTION

As used herein, the term “hydrocarbon group” (also denoted by the group R) is defined as a chemical group composed of at least carbon and hydrogen. In some embodiments, the hydrocarbon group may (i.e., optionally) be substituted with one or more fluorine atoms to result in partial or complete fluorination of the hydrocarbon group, and/or the hydrocarbon group may or may not also contain a single ether or thioether linkage connecting between carbon atoms in the hydrocarbon group. The hydrocarbon group typically contains 1-30 carbon atoms. In different embodiments, one or more of the hydrocarbon groups may contain, for example, 1, 2, 3, 4, 5, 6, 7, 8, 9, 10, 12, 15, 18, 20, 22, 24, 26, 28, or 30 carbon atoms, or a number of carbon atoms within a particular range bounded by any two of the foregoing carbon numbers (e.g., 1-30, 2-30, 3-30, 4-30, 6-30, 8-30, 10-30, 12-30, 1-20, 6-20, 8-20, 10-20, 12-20, 1-12, 3-12, 6-12, or 8-12 carbon atoms). Hydrocarbon groups in different compounds described herein, or in different positions of a compound, may possess the same or different number (or preferred range thereof) of carbon atoms in order to independently adjust or optimize such properties as the complexing ability, extracting (extraction affinity) ability, selectivity ability, or third phase prevention ability of the compound.

In a first set of embodiments, the hydrocarbon group (R) is a saturated and straight-chained group, i.e., a straight-chained (linear) alkyl group. Some examples of straight-chained alkyl groups include methyl, ethyl, n-propyl, n-butyl, n-pentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-eicosyl, n-docosyl, n-tetracosyl, n-hexacosyl, n-octacosyl, and n-triacontyl groups.

In a second set of embodiments, the hydrocarbon group (R) is saturated and branched, i.e., a branched alkyl group. Some examples of branched alkyl groups include isopropyl, isobutyl(2-methylprop-1-yl), sec-butyl(2-butyl), t-butyl (1,1-dimethylethyl-1-yl), 2-pentyl, 3-pentyl, 2-methylbut-1-yl, isopentyl (3-methylbut-1-yl), 1,2-dimethylprop-1-yl, 1,1-dimethylprop-1-yl, neopentyl (2,2-dimethylprop-1-yl), 2-hexyl, 3-hexyl, 2-methylpent-1-yl, 3-methylpent-1-yl, isohexyl (4-methylpent-1-yl), 1,1-dimethylbut-1-yl, 1,2-dimethylbut-1-yl, 2,2-dimethylbut-1-yl, 2,3-dimethylbut-1-yl, 3,3-dimethylbut-1-yl, 1,1,2-trimethylprop-1-yl, 1,2,2-trimethylprop-1-yl, isoheptyl, isooctyl, and the numerous other branched alkyl groups having up to 20 or 30 carbon atoms, wherein the “1-yl” suffix represents the point of attachment of the group.

In a third set of embodiments, the hydrocarbon group (R) is saturated and cyclic, i.e., a cycloalkyl group. Some examples of cycloalkyl groups include cyclopropyl, cyclobutyl, cyclopentyl, cyclohexyl, cycloheptyl, and cyclooctyl groups. The cycloalkyl group can also be a polycyclic (e.g., bicyclic) group by either possessing a bond between two ring groups (e.g., dicyclohexyl) or a shared (i.e., fused) side (e.g., decalin and norbornane).

In a fourth set of embodiments, the hydrocarbon group (R) is unsaturated and straight-chained, i.e., a straight-chained (linear) olefinic or alkenyl group. The unsaturation occurs by the presence of one or more carbon-carbon double bonds and/or one or more carbon-carbon triple bonds. Some examples of straight-chained olefinic groups include vinyl, propen-1-yl (allyl), 3-buten-1-yl (CH₂═CH—CH₂—CH₂—), 2-buten-1-yl (CH₂—CH═CH—CH₂—), butadienyl, 4-penten-1-yl, 3-penten-1-yl, 2-penten-1-yl, 2,4-pentadien-1-yl, 5-hexen-1-yl, 4-hexen-1-yl, 3-hexen-1-yl, 3,5-hexadien-1-yl, 1,3,5-hexatrien-1-yl, 6-hepten-1-yl, ethynyl, propargyl(2-propynyl), 3-butynyl, and the numerous other straight-chained alkenyl or alkynyl groups having up to 20 or 30 carbon atoms.

In a fifth set of embodiments, the hydrocarbon group (R) is unsaturated and branched, i.e., a branched olefinic or alkenyl group. Some examples of branched olefinic groups include 1-buten-2-yl (CH₂═C·—CH₂—CH₃), 1-buten-3-yl (CH₂—CH—CH·—CH₃), 1-propen-2-methyl-3-yl (CH₂—C(CH₃)—CH₂—), 1-penten-4-yl, 1-penten-3-yl, 1-penten-2-yl, 2-penten-2-yl, 2-penten-3-yl, 2-penten-4-yl, and 1,4-pentadien-3-yl, and the numerous other branched alkenyl groups having up to 20 or 30 carbon atoms, wherein the dot in any of the foregoing groups indicates a point of attachment.

In a sixth set of embodiments, the hydrocarbon group (R) is unsaturated and cyclic, i.e., a cycloalkenyl group. The unsaturated cyclic group may be aromatic or aliphatic. Some examples of unsaturated cyclic hydrocarbon groups include cyclobutenyl, cyclopentenyl, cyclopentadienyl, cyclohexenyl, cyclohexadienyl, phenyl, benzyl, cycloheptenyl, cycloheptadienyl, cyclooctenyl, cyclooctadienyl, and cyclooctatetraenyl groups. The unsaturated cyclic hydrocarbon group may or may not also be a polycyclic group (such as a bicyclic or tricyclic polyaromatic group) by either possessing a bond between two of the ring groups (e.g., biphenyl) or a shared (i.e., fused) side, as in naphthalene, anthracene, phenanthrene, phenalene, or indene fused ring systems.

As indicated earlier above, any of the hydrocarbon groups described above may be substituted with one or more fluorine atoms. As an example, an n-octyl group may be substituted with a single fluorine atom to result in, for example, a 7-fluorooctyl or 8-fluorooctyl group, or substituted with two or more fluorine atoms to result in, for example, 7,8-difluorooctyl, 8,8-difluorooctyl, 8,8,8-trifluorooctyl, or perfluorooctyl group. As also indicated earlier above, any of the hydrocarbon groups described above may (or may not) contain a single ether (—O—) or thioether (—S—) linkage connecting between carbon atoms in the hydrocarbon group. An example of a hydrocarbon group containing a single ether or thioether group is —(CH₂) 2-X—(CH₂) 7CH₃, wherein X represents O or S. Moreover, an aromatic or aliphatic ring may contain one or more ring nitrogen and/or oxygen atoms, such as in the heterocyclic groups pyridine, piperidine, piperazine, pyrrole, imidazole, pyrazine, pyrimidine, triazole, oxazole, morpholine, furan, tetrahydrofuran, and dioxane.

In one aspect, the present disclosure is directed to hydrophobic metal chelator compounds (i.e., “rare earth extractant compounds” or “rare earth complexing agents”) having an ability to complex more strongly or more favorably (more selectively) with lower atomic or larger radii lanthanide elements compared to higher atomic or smaller radii lanthanide elements. The hydrophobic metal chelator compounds are within the scope of the following generic structure:

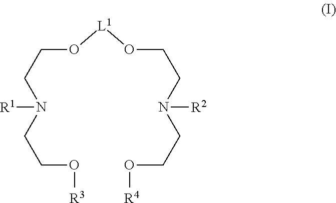

The variable L¹ in Formula (I) is a linker containing 2-8 carbon atoms optionally containing one or more heteroatoms selected from O, N, and S atoms. The linker L¹ can be derived from any of the hydrocarbon groups described above containing 2-8 carbon atoms by removal of a hydrogen atom. For example, L¹ can be an ethylene (—CH₂CH₂—) linker by removal of a hydrogen atom from an ethyl group, or L¹ can be a propylene (—CH₂CH₂CH₂—) linker by removal of a hydrogen atom from a propyl group, or L¹ can be an ethylene-oxy-ethylene (—CH₂CH₂—O—CH₂CH₂—) linker by removal of a hydrogen atom from an ethylene-oxy-ethyl (—CH₂CH₂—O—CH₂CH₃) group, or L¹ can be a phenylene (—C₆CH₄—) linker by removal of a hydrogen atom from a phenyl (—C₆H₅) group. In some embodiments, L¹ is selected from a linker of the formula (CH₂) p, wherein p is an integer of 2, 3, 4, 5, 6, 7, or 8 or an integer within a range bounded by any two of these values, e.g., 2-6, 2-4, 2-3, 3-8, 3-6, 3-4, 4-8, 4-6, 5-8, 5-6, or 6-8. In any of the foregoing examples of (CH₂) p linkers, one or more hydrogen atoms may be substituted with one or more methyl groups to result in a branched linker (e.g., —CH(CH₃) CH₂- or —CH(CH₃) CH(CH₃)—). In further or separate embodiments, any of the foregoing linear or branched (CH₂) p linkers may contain one or more heteroatoms or heteroatom-containing groups (e.g., O, S, NH, or N(CH₃)) interrupting a carbon-carbon bond (e.g., (—CH₂CH₂—O—CH₂CH₂—, —CH₂CH₂—S—CH₂CH₂—, —CH₂CH₂—NH—CH₂CH₂—, or —CH₂CH₂—N(CH₃)—CH₂CH₂—). In other embodiments, L¹ is a cyclic linker, wherein the cyclic linker can be derived from any of the carbocyclic or heterocyclic groups described earlier above, wherein the cyclic linker typically connects via two of its ring carbon atoms. The linker L¹ may also contain a linear or branched hydrocarbon linking portion connected to a ring linking portion, such as in-CH₂-phenylene-CH₂—.

The variables R¹ and R² in Formula (I) are independently selected from hydrocarbon groups containing 1-12 carbon atoms, such as any of those described above, optionally containing one or more heteroatoms selected from O, N, and S atoms, with each one of R¹ and R² being substituted with at least one heteroatom-containing group selected from carboxy groups of the formula —C(O) OR′; phosphoryl groups of the formula —P(—O)(OH)R″; oxy groups of the formula OR′; amino groups of the formula —NR′₂; and amido groups of the formula —C(O)NHR′₂. In the above groups, R′ is independently selected from H and hydrocarbon groups containing 1-6 carbon atoms and R″ is selected from OH and hydrocarbon groups containing 1-6 carbon atoms. In some embodiments, R¹ and R² each include a carbocyclic group, such as any those described above (e.g., phenyl). In other embodiments, R¹ and R² each include a heterocyclic group, such as any of those described above, e.g., pyridine, piperidine, piperazine, pyrrole, imidazole, pyrazine, pyrimidine, triazole, oxazole, morpholine, furan, tetrahydrofuran, and dioxane. The heterocyclic ring may be directly attached to the nitrogen atom in the macrocycle portion of formula (I) typically by a ring carbon atom, or more typically, the heterocyclic ring is attached to the nitrogen atom of the macrocycle portion of Formula (I) via a linkage, such as a heteroatom linker (e.g., —O— or —S—), heteroatom group linker (e.g., —NH—, —NH(CH₃)—, or —C(O) NH-linker) or a linker of the formula (CH₂) t, wherein t is an integer of 1, 2, or 3 or an integer within a range bounded by any two of these values and wherein (CH₂) t may be substituted with a methyl group and/or contain a heteroatom selected from O, N, or S. The linkage is typically attached to a ring carbon atom of the heterocyclic ring, wherein the ring carbon atom is typically adjacent to a ring nitrogen, oxygen, or sulfur atom. In more particular embodiments, R¹ and R² are selected from nitrogen-containing rings, such as pyridine, pyrimidine, triazole, piperidine, or piperazine rings, and more particularly, wherein any such rings are substituted with any of the above carboxy, phosphoryl, oxy, amino, or amido groups at a ring carbon atom adjacent to (i.e., connecting to) a ring nitrogen atom. Typically, R¹ and R² are the same.

In some embodiments, R¹ and R² are independently selected from the following groups:

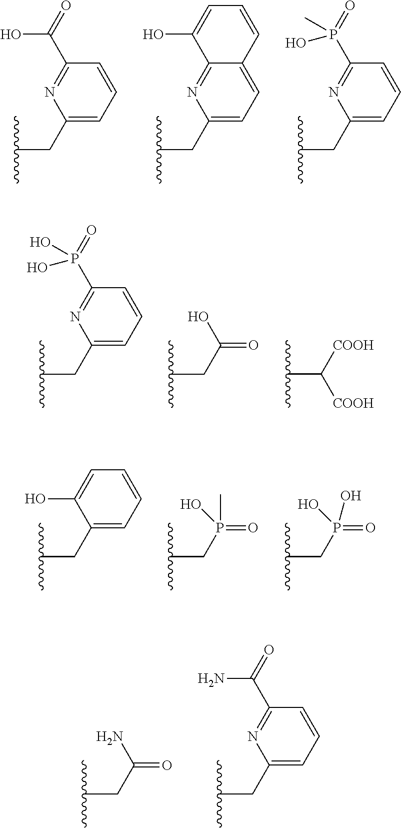

Typically, R¹ and R² are the same. Moreover, in embodiments, any generic or specific selection of L¹, as provided above, may be made and combined with any generic or specific selection of R¹ and R² as provided above.

The variables R³ and R⁴ in Formula (I) are independently selected from hydrocarbon groups, such as any of those described earlier above, containing 4-30 carbon atoms. The hydrocarbon groups containing 4-30 carbon atoms may be or include any of the linear or branched alkyl or alkenyl groups or cyclic groups described above containing 4-30 carbon atoms. The hydrocarbon groups for R³ and R⁴ may also be a combination of an alkyl or alkenyl group or linker attached to a carbocyclic or heterocyclic group or linker. In some embodiments, R³ and R⁴ may contain solely carbon and hydrogen atoms while in other embodiments R³ and R⁴ may (or may not) be partially or fully substituted with fluorine atoms and/or R³ and R⁴ may (or may not) contain a single O, S, NH, or N(CH₃) linkage interrupting a carbon-carbon bond. In different embodiments, R³ and R⁴ may independent contain 1, 2, 3, 4, 5, 6, 7, 8, 9, 10, 12, 15, 18, 20, 22, 24, 26, 28, or 30 carbon atoms, or a number of carbon atoms within a particular range bounded by any two of the foregoing carbon numbers (e.g., 4-30, 5-30, 6-30, 7-30, 8-30, 9-30, 10-30, 12-30, 4-20, 5-20, 6-20, 7-20, 8-20, 9-20, 10-20, 12-20, 4-12, 6-12, or 8-12 carbon atoms).

In some embodiments, the compound of Formula (I) has the following more particular structure in which L¹ is selected as an ethylene linker:

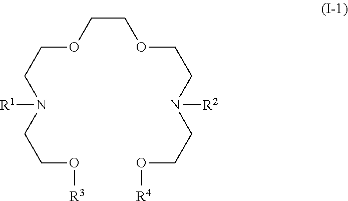

wherein R¹, R², R³, and R⁴ are as defined above under Formula (I). Any selections of R¹, R², R³, and R⁴, as defined and described above under Formula (I), may be made and combined to arrive at one or more compounds within the scope of Formula (I-1). In particular embodiments of Formula (I-1), R³ and R⁴ contain 4-30, 5-30, 6-30, 7-30, 8-30, 9-30, 10-30, 12-30, 4-20, 5-20, 6-20, 7-20, 8-20, 9-20, 10-20, 12-20, 4-12, 6-12, or 8-12 carbon atoms.

In some embodiments, the compound of Formula (I) has the following more particular structure in which L¹ is selected as an ethylene-oxy-ethylene linker:

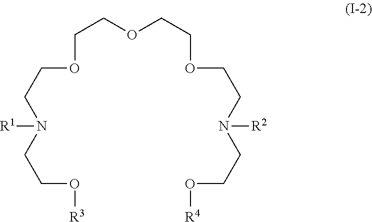

wherein R¹, R², R³, and R⁴ are as defined above under Formula (I). Any selections of R¹, R², R³, and R⁴, as defined and described above under Formula (I), may be made and combined to arrive at one or more compounds within the scope of Formula (I-2). In particular embodiments of Formula (I-2), R³ and R⁴ contain 4-30, 5-30, 6-30, 7-30, 8-30, 9-30, 10-30, 12-30, 4-20, 5-20, 6-20, 7-20, 8-20, 9-20, 10-20, 12-20, 4-12, 6-12, or 8-12 carbon atoms.

In some embodiments, the compound of Formula (I) has the following more particular structure in which L¹ is selected as an o-phenylene linker:

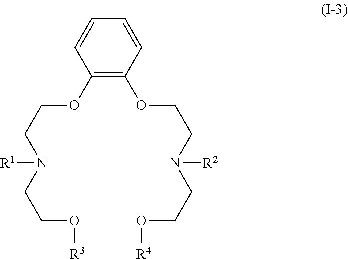

wherein R¹, R², R³, and R⁴ are as defined above under Formula (I). Any selections of R¹, R², R³, and R⁴, as defined and described above under Formula (I), may be made and combined to arrive at one or more compounds within the scope of Formula (I-3). In particular embodiments of Formula (1-3), R³ and R⁴ contain 4-30, 5-30, 6-30, 7-30, 8-30, 9-30, 10-30, 12-30, 4-20, 5-20, 6-20, 7-20, 8-20, 9-20, 10-20, 12-20, 4-12, 6-12, or 8-12 carbon atoms.

In some embodiments, the compound of Formula (I) has the following structure:

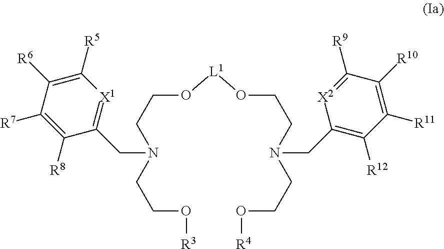

The variables L¹, R³, and R⁴ in Formula (Ia) are as defined and exemplified above under Formula (I). Any selections and combinations of these variables described above are considered for the structure in Formula (Ia).

The variables R⁵, R⁶, R⁷, R⁸, R⁹, R¹⁰, R¹¹, and R¹² in Formula (Ia) are independently selected from H, hydrocarbon groups containing 1-3 carbon atoms; carboxy groups of the formula —C(O) OR′; phosphoryl groups of the formula —P(═O)(OH)R″; oxy groups of the formula OR′; amino groups of the formula —NR′₂; and amido groups of the formula —C(O)NHR′₂. In the foregoing groups, R′ is independently selected from H and hydrocarbon groups containing 1-6 carbon atoms and R″ is selected from OH and hydrocarbon groups containing 1-6 carbon atoms. In some embodiments, a pair of adjacent groups selected from R⁵, R⁶, R⁷, and R⁸ (e.g., R⁵ and R⁶, or R⁶ and R⁷, or R⁷ and R⁸) and/or a pair of adjacent groups selected from R⁹, R¹⁰, R¹¹, and R¹² (e.g., R⁹ and R¹⁰, or R¹⁰ and R¹¹, or R¹¹ and R¹²) are optionally interconnected to form an additional ring to form a fused ring system, wherein the additional ring may optionally and independently be substituted with one or more hydrocarbon groups containing 1-3 carbon atoms; carboxy groups of the formula —C(O) OR′; phosphoryl groups of the formula —P(═O)(OH)R″; oxy groups of the formula OR′; amino groups of the formula —NR′₂; and amido groups of the formula —C(O)NHR′₂; wherein R′ is independently selected from H and hydrocarbon groups containing 1-6 carbon atoms and R″ is selected from OH and hydrocarbon groups containing 1-6 carbon atoms. In some embodiments, at least one of R⁵, R⁶, R⁷, and R⁸ is selected from carboxy, phosphoryl, oxy, amino, and amido groups (or more particularly carboxy or phosphoryl groups), and at least one of R⁹, R¹⁰, R¹¹, and R¹² is selected from carboxy, phosphoryl, oxy, amino, and amido groups (or more particularly carboxy or phosphoryl groups). In some embodiments, only R⁵ and R° are selected from any of the carboxy, phosphoryl, oxy, amino, or amido groups described above while the remaining R⁶, R⁷, R⁸, R¹⁰, R¹¹, and R¹² groups are H. In more particular embodiments, only R⁵ and R° are selected from carboxy or phosphoryl groups while the remaining R⁶, R⁷, R⁸, R¹⁰, R¹¹, and R¹² groups are H.

The variable X¹ in Formula (Ia) is selected from CR^{a} and N, wherein R^{a} is selected from hydrocarbon groups containing 1-3 linearly connected carbon atoms; carboxy groups of the formula —C(O) OR′; phosphoryl groups of the formula —P(═O)(OH)R″; oxy groups of the formula OR′; amino groups of the formula —NR′₂; and amido groups of the formula —C(O)NHR′₂; wherein R′ is independently selected from H and hydrocarbon groups containing 1-6 carbon atoms and R″ is selected from OH and hydrocarbon groups containing 1-6 carbon atoms. Similarly, X² in Formula (Ia) is selected from CR^{b} and N, wherein R^{b} is selected from hydrocarbon groups containing 1-3 linearly connected carbon atoms; carboxy groups of the formula —C(O) OR′; phosphoryl groups of the formula —P(═O)(OH)R″; oxy groups of the formula OR′; amino groups of the formula —NR′₂; and amido groups of the formula —C(O)NHR′₂; wherein R′ is independently selected from H and hydrocarbon groups containing 1-6 carbon atoms and R″ is selected from OH and hydrocarbon groups containing 1-6 carbon atoms. Notably, at least one of R^{a}, R⁵, R⁶, R⁷, and R⁸ is selected from carboxy, phosphoryl, oxy, amino, and amido groups, and at least one of R^{b}, R⁹, R¹⁰, R¹¹, and R¹² is selected from carboxy, phosphoryl, oxy, amino, and amido groups. In the event that R^{a} and R^{b} are both selected from carboxy, phosphoryl, oxy, amino, and amido groups, R⁵, R⁶, R⁷, R⁸, R⁹, R¹⁰, R¹¹, and R¹² may not be any of these groups (e.g., may be selected from hydrocarbon groups and H atom) or one or more of R⁵, R⁶, R⁷, R⁸, R⁹, R¹⁰, R¹¹, and R¹² may also include one or more of carboxy, phosphoryl, oxy, amino, and amido groups. Any selections of variables L¹, R³, and R⁴ in Formula (Ia) as defined and exemplified above under Formula (I) may be made and combined with any selection of variables R⁵, R⁶, R⁷, R⁸, R⁹, R¹⁰, R¹¹, and R¹² in Formula (Ia) and this may further be combined with any selection of X¹ and X² in Formula (Ia), as provided above, to result in one or more compounds within the scope of Formula (Ia).

Some particular types of molecules within the scope of Formula (Ia) include the following:

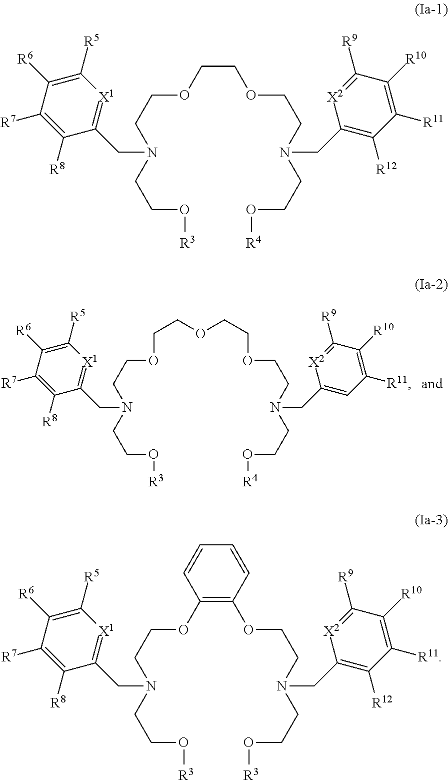

In some embodiments, the extractant molecule has any of the above more particular structures. The variables R³, R⁴, R⁵, R⁶, R⁷, R⁸, R⁹, R¹⁰, R¹¹, R¹², X¹, and X² in Formulas (Ia-1), (Ia-2), and (Ia-3) are as defined and exemplified above under Formulas (I) and (Ia). Any selections and combinations of these variables described above can be made to result in one or more compounds within the scope of Formula (Ia-1), (Ia-2), or (Ia-3). In specific embodiments of any of Formulas (I), (Ia), (Ia-1), (Ia-2), and (Ia-3), R³ and R⁴ are independently selected from hydrocarbon groups containing 5-30, 6-30, 7-30, 8-30, 9-30, 10-30, 11-30, or 12-30 carbon atoms.

A more particular class of molecules within the scope of Formula (Ia) has the following structure:

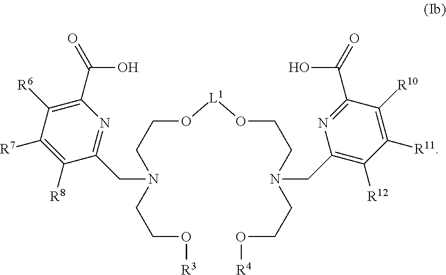

The variables L¹, R³, and R⁴ in Formula (Ia) are as defined and exemplified above under Formula (I). Any selections and combinations of these variables described above are considered for the structure in Formula (Ia). The variables R⁶, R⁷, R⁸, R¹⁰, R¹¹, and R¹² are as defined and exemplified above under Formula (Ia). Any selections and combinations of these variables provided above may be made to arrive at one or more compounds within the scope of Formula (Ib).

Some particular types of molecules within the scope of Formula (Ib) include the following:

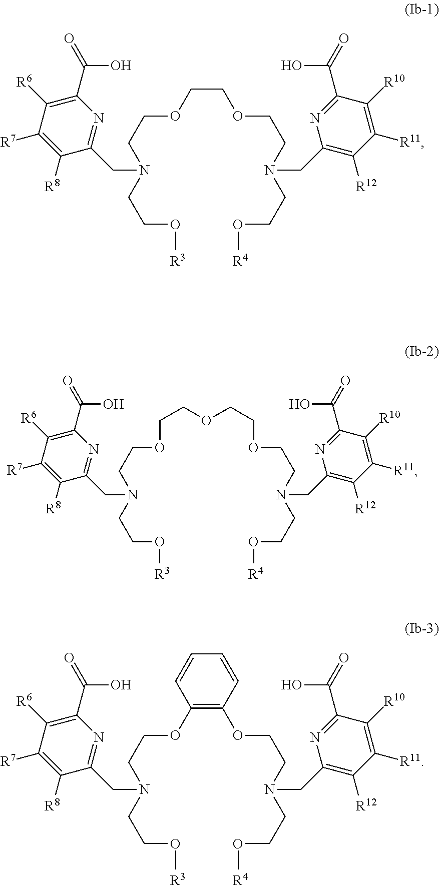

In some embodiments, the extractant molecule has any of the above more particular structures. The variables R³, R⁴, R⁶, R⁷, R⁸, R¹⁰, R¹¹, and R¹² in Formulas (Ib-1), (Ib-2), and (Ib-3) are as defined and exemplified above under Formulas (Ia) and (Ib). Any selections and combinations of these variables described above can be made to arrive at one or more compounds within the scope of Formula (Ib-1), (Ib-2), or (Ib-3). In specific embodiments of any of Formulas (Ib), (Ib-1), (Ib-2), and (Ib-3), R³ and R⁴ are independently selected from hydrocarbon groups containing 5-30, 6-30, 7-30, 8-30, 9-30, 10-30, 11-30, or 12-30 carbon atoms.

In some embodiments, the extractant molecule has the following specific chemical structure:

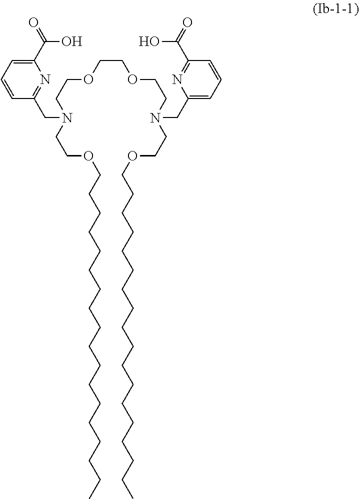 [SMILES: CCCCCCCCCCCCCCCCCCOCCN(CCOCCOCCN(CCOCCCCCCCCCCCCCCCCCC)Cc1cccc(C(=O)O)n1)Cc1cccc(C(=O)O)n1]

In some embodiments, the extractant molecule has the following specific chemical structure:

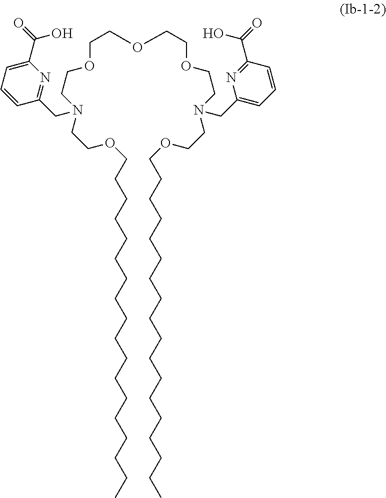 [SMILES: CCCCCCCCCCCCCCCCCCOCCN(CCOCCOCCOCCN(CCOCCCCCCCCCCCCCCCCCC)Cc1cccc(C(=O)O)n1)Cc1cccc(C(=O)O)n1]

In some embodiments, the extractant molecule has the following specific chemical structure:

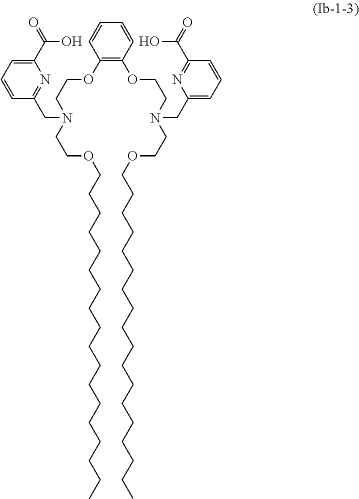 [SMILES: CCCCCCCCCCCCCCCCCCOCCN(CCOc1ccccc1OCCN(CCOCCCCCCCCCCCCCCCCCC)Cc1cccc(C(=O)O)n1)Cc1cccc(C(=O)O)n1]

Compounds of Formula (I) and sub-formulas thereof can be synthesized by means well known in the art, as further described in the Examples section. For example, precursor compounds according to Formula (I) can first be prepared in which R¹ and R² are benzyl groups and R³ and R⁴ are any of the hydrocarbon groups described above containing at least four carbon atoms, followed by removal of the benzyl groups by hydrogen reduction, followed by reaction of the two resulting NH linkages with an alkyl-brominated molecule containing R¹ and R² moieties, to result in a molecule of interest within the scope of Formula (I). The above general procedure can be used to make a wide variety of compounds within the scope of Formula (I), including compounds in which R¹ and R² are substituted with at least one heteroatom-containing group selected from: carboxy groups of the formula —C(O) OR′; phosphoryl groups of the formula —P(═O)(OH)R″; oxy groups of the formula OR′; amino groups of the formula —NR′₂; and amido groups of the formula —C(O)NHR′₂; wherein R′ is independently selected from H and hydrocarbon groups containing 1-6 carbon atoms and R″ is selected from OH and hydrocarbon groups containing 1-6 carbon atoms; and compounds in which R³ and R⁴ are independently selected from hydrocarbon groups containing 4-30 carbon atoms, wherein R³ and R⁴ are optionally partially or fully fluorinated and/or optionally contain a single O, S, NH, or N(CH₃) linkage interrupting a carbon-carbon bond.

In another aspect, the present disclosure is directed to a hydrophobic extractant solution useful for more preferentially extracting lower atomic number or larger radii RE element(s) from aqueous solutions containing a mixture of lanthanide elements. The hydrophobic extractant solution is aqueous-insoluble, and thus, insoluble with an acidic or buffered aqueous solution containing the mixture of lanthanide elements. The aqueous-insoluble hydrophobic solution contains, at minimum, a lipophilic extractant compound, selected from any of those described above within the scope of Formula (I) or sub-formula thereof, dissolved in an aqueous-insoluble hydrophobic solvent. Any one or more of the extractant compounds within the scope of Formula (1) or any sub-formula thereof can function as a lipophilic extractant compound and be included in the aqueous-insoluble hydrophobic solution. The one or more lipophilic extractant compounds may be present in the hydrophobic extractant solution in a concentration of, for example, precisely, at least, or up to, for example, 0.01 M, 0.02 M, 0.05 M, 0.1 M, 0.2 M, 0.3 M, 0.4 M, 0.5 M, 0.6 M, 0.7 M, 0.8 M, 0.9 M, or 1 M or a concentration within a range bounded by any two of the foregoing values, e.g., 0.01-1 M, 0.01-0.5 M, 0.05-1 M, 0.05-0.5 M, 0.1-1 M, 0.1-0.8 M, 0.1-0.5 M, 0.2-1 M, 0.2-0.8 M, or 0.2-0.5 M. In the case where more than one extractant compound is included in the hydrophobic solution, any two or more of the above described extractant compounds may be selected, and each one may be independently included in any of the exemplary concentrations above, and/or the extractant compounds may have a total (combined) concentration corresponding to any of the above exemplary concentrations.

The aqueous-insoluble hydrophobic solvent may be any solvent that is substantially or completely insoluble (immiscible) in water or aqueous solutions and which also fully dissolves the RE extractant compound. The aqueous-insoluble hydrophobic solvent may be, for example, a hydrocarbon solvent, ether solvent, or alcohol solvent containing at least six carbon atoms. In some embodiments, the hydrophobic solvent is composed solely of carbon and hydrogen. In other embodiments, the hydrophobic solvent optionally includes one or more halogens (e.g., one or more fluorine or chlorine atoms). The hydrophobic solvent typically has a melting point up to or below 0° C. or −10° C. Some examples of hydrocarbon solvents include non-halogenated types (e.g., hexanes, heptanes, octanes, decanes, dodecanes, benzene, toluene, xylenes, kerosene, decalin, or petroleum ether) and halogenated types (e.g., methylene chloride, chloroform, carbon tetrachloride, 1,2-dichlorethane, tetrachloroethane, trichloroethylene, perchloroethylene, hexafluorobenzene, octafluorotoluene, and tetradecafluoro-2-methylpentane). Some examples of ether solvents include diethyl ether and diisopropyl ether. Some examples of alcohol solvents containing at least six carbon atoms include 1-hexanol, 2-hexanol, 3-hexanol, perfluorohexanol, 1-heptanol, 1-octanol, isooctyl alcohol (2-ethylhexanol), perfluorooctanol, 1-nonanol, 1-decanol, 1-undecanol, 1-dodecanol, cyclohexanol, and 2-methylcyclohexanol. The hydrophobic solvent may also have a combination of halogen and ether groups, such as in bis(chloroethyl) ether, 2-chloroethyl vinyl ether, and 1,1,1,2,2,3,3-heptafluoro-3-methoxypropane.

In some embodiments, the hydrophobic extractant solution is composed solely of one or more lipophilic extractant compound(s) and the aqueous-insoluble hydrophobic solvent. In other embodiments, the hydrophobic extractant solution contains one or more additional components, such as any of those described below.

In some embodiments, the hydrophobic extractant solution, described above, further includes an organoamine soluble in the aqueous-insoluble hydrophobic solvent. The organoamine may function to, for example, further bind to the rare earth element (REE), prevent or lessen formation of a third phase during the extraction, and/or assist in removing (stripping) the REE from the aqueous-insoluble hydrophobic solvent after extraction. To be soluble in the hydrophobic solvent, the organoamine should be sufficiently hydrophobic (lipophilic). To be sufficiently hydrophobic, the organoamine should contain at least one hydrocarbon group containing at least four carbon atoms. However, to ensure full solubility of the organoamine in the hydrophobic solvent, the organoamine preferably contains, in total, at least or more than six carbon atoms. In different embodiments, the organoamine may contain at least or more than, for example, 6, 7, 8, 9, 10, 11, 12, 13, 14, 15, 16, 17, 18, 19, or 20 carbon atoms, or a number of carbon atoms within a range bounded by any two of the foregoing values. The organoamine may be a primary, secondary, or tertiary amine. Some examples of primary organoamines include n-hexylamine, isohexylamine, n-heptylamine, n-octylamine, isooctylamine, n-nonylamine, n-decylamine, n-undecylamine, n-dodecylamine, n-tridecylamine, n-tetradecylamine, and n-hexadecylamine. Some examples of secondary organoamines include dibutylamine, diisobutylamine, dipentylamine, dihexylamine, diheptylamine, diooctylamine, dinonylamine, didecylamine, didodecylamine, N-methylbutylamine, N-methylpentylamine, N-methylhexylamine, N-methylheptylamine, N-methyloctylamine, N-ethylbutylamine, and N-ethyloctylamine. Some examples of tertiary organoamines include tributylamine, tripentylamine, trihexylamine, triheptylamine, trioctylamine, trinonylamine, tridecylamine, triundecylamine, and tridodecylamine. In some embodiments, any one or more of the above amines, or any amine altogether, is/are excluded from the hydrophobic extractant solution.

In some embodiments, the hydrophobic extractant solution, as described above, further includes an organoamide soluble in the aqueous-insoluble hydrophobic solvent. The organoamide may function to, for example, further bind to the REE, prevent formation of a third phase during the extraction, and/or assist in removing (stripping) the REE from the aqueous-insoluble hydrophobic solvent after extraction. To be soluble in the hydrophobic solvent, the organoamide should be sufficiently hydrophobic (lipophilic). To be sufficiently hydrophobic, the organoamide should contain at least one hydrocarbon group containing at least four carbon atoms. However, to ensure full solubility of the organoamide in the hydrophobic solvent, the organoamide preferably contains, in total, at least or more than six carbon atoms. In different embodiments, the organoamide may contain at least or more than, for example, 6, 7, 8, 9, 10, 11, 12, 13, 14, 15, 16, 17, 18, 19, or 20 carbon atoms, or a number of carbon atoms within a range bounded by any two of the foregoing values. Some examples of hydrophobic organoamides include N-methylpentanamide, N-ethylpentanamide, N-propylpentanamide, N-butylpentanamide, N-pentylpentanamide, N-hexylpentanamide, N-methylhexanamide, N-ethylhexanamide, N-propylhexanamide, N-methyloctanamide, N-ethyloctanamide, N-propyloctanamide, N-methyldecanamide, N-ethyldecanamide, N-propyldecanamide, N,N-dimethylpentanamide, N,N-diethylpentanamide, N,N-dipropylpentanamide, N,N-dibutylpentanamide, N,N-dihexylpentanamide, and N,N-diethyloctanamide. In some embodiments, any one or more of the above amides, or any amide altogether, is/are excluded from the hydrophobic extractant solution.

In some embodiments, the hydrophobic extractant solution, as described above, further includes an alcohol soluble in the aqueous-insoluble hydrophobic solvent. The alcohol may or may not correspond to one of the hydrophobic alcohol solvents containing at least six carbon atoms described above. The alcohol generally functions to prevent or lessen formation of a third phase during the extraction. To be soluble in the hydrophobic solvent, the alcohol should be sufficiently hydrophobic (lipophilic). To be sufficiently hydrophobic, the alcohol typically contains at least or more than six carbon atoms. In different embodiments, the alcohol contains at least or more than, for example, 6, 7, 8, 9, 10, 11, 12, 13, 14, 15, 16, 17, 18, 19, or 20 carbon atoms, or a number of carbon atoms within a range bounded by any two of the foregoing values. Some examples of lipophilic alcohols include n-hexyl alcohol, 4-methyl-1-pentanol, n-heptanol, n-octanol, 6-methyl-1-heptanol, 2-ethyl-1-hexanol, n-decanol, n-dodecanol, n-tridecanol, isotridecanol, n-tetradecanol, and n-hexadecanol. In some embodiments, any one or more of the above alcohols, or any alcohol altogether, is/are excluded from the hydrophobic extractant solution.

In another aspect, the present disclosure is directed to metal-ligand complexes containing a lipophilic extractant compound (i.e., metal chelator compound), such as any of those described above within the scope of Formula (I) or sub-formula thereof, and a metal (M) complexed with the lipophilic extractant compound. In the complex, the metal is typically bonded to two or more of the nitrogen and/or oxygen atoms in the macrocyclic portion of the extractant compound. The metal atom may additionally be bonded to one or more groups residing in R¹ and R² of the extractant compound. The metal may or may not be additionally bound to other ligands, such as solvent molecules and any of the common anions (e.g., halide, oxide, carbonate, sulfate, nitrate, phosphate, and the like). The metal-ligand complex may be a result of extracting the metal from an aqueous solution and may be useful as an isolated source of the metal wherein the ligand portion may be removed by suitable means to provide a pure source of the metal.

The metal (M) in the metal-ligand complex may be, for example, a transition metal element, lanthanide element, actinide element, alkali element, or alkaline earth element. Some examples of transition metals include those within Groups 3-11 or 3-12 of the Periodic Table (e.g., Sc, Ti, V, Cr, Mn, Fe, Co, Ni, Cu, Zn, Y, Zr, Nb, Mo, Tc, Ru, Rh, Pd, Hf, Ta, W, Pt, or Au, or a subset thereof). The term “lanthanide element,” as used herein, refers to those elements having an atomic number of 57-71. The lanthanide elements are listed as follows: lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), and lutetium (Lu). The term “actinide element,” as used herein, refers to those elements having an atomic number of 90-103. Some examples of actinide elements include actinium, thorium, uranium, neptunium, plutonium, and americium. Some examples of alkali elements include Li, Na, K, R^{b}, and Cs. Some examples of alkaline earth elements include Mg, Ca, Sr, Ba, and R^{a}. In some embodiments, the metal is a rare earth element, which includes the lanthanide elements along with Sc and/or Y. Moreover, if A represents the ligand and M the metal, any molar stoichiometry between A and M in the complex is contemplated, such as MA, M₂A, MA₂, and the like.

In another aspect, the present disclosure is directed to a method of at least partially separating one rare earth (RE) element from another from an aqueous solution containing at least two RE elements. In the method, the above aqueous solution is contacted with an aqueous-insoluble hydrophobic solution, as described above, containing a rare earth extractant compound dissolved in an aqueous-insoluble hydrophobic solvent. As the hydrophobic solution is immiscible with the aqueous solution, the two solutions cannot dissolve into each other and will remain separated during and after contact. The two solutions can be contacted by any means that result in intimate mixing, such as by shaking followed by standing to allow the two phases to separate after mixing. In some embodiments, the liquids are intimately mixed by subjecting them to vortex mixing. Following mixing, the two phases can be separated by means well known in the art, such as by standing or centrifugation. The result of the contacting step is that the aqueous-insoluble hydrophobic solution contains a higher molar ratio of lower atomic number or larger radii RE element(s) over higher atomic number or smaller radii RE element(s) compared to the corresponding molar ratio (i.e., of lower atomic number or larger radii RE element(s) over higher atomic number or smaller radii RE element(s)) in the aqueous solution. More particularly, the method may achieve at least partial selective extraction into the aqueous-insoluble hydrophobic solution of one or more lighter lanthanide element(s) having an atomic number in a range of 57-65 over one or more heavier lanthanide element(s) having an atomic number in a range of 66-71, wherein the aqueous solution contains at least one element selected from said lighter lanthanide elements and at least one element selected from said heavier lanthanide elements. Any one or more of the extractant compounds described above within the scope of Formula (1) or sub-formula therein may be included in the aqueous-insoluble hydrophobic solution.

The at least two RE elements in the aqueous solution can be selected from any of the RE elements provided above. In some embodiments, the RE elements include adjacent lanthanide elements, wherein the term “adjacent lanthanide elements” refers to lanthanide elements differing by one atomic number, such as Nd/Pr, Eu/Sm, Nd/Pm, or Tb/Gd pairs. In separate or further embodiments, the RE elements include at least two lanthanide elements separated by precisely or at least two, three, or more atomic numbers, and may or may not include Sc and/or Y. In the aqueous solution, the RE elements are present in ionic form (e.g., Nd⁺3), which is typically a salt form (e.g., Nd₂ (SO₄)₃) or complexed form. The aqueous solution may or may not also contain at least one (or one or more) of any of the actinide elements, such as uranium (U) and/or thorium (Th). For purposes of the present invention, the aqueous solution is typically not acidified, particularly not with a strong acid, such as a mineral acid (e.g., hydrochloric, nitric, or sulfuric acid).

In some embodiments, the aqueous solution further contains a buffer that maintains the aqueous solution in a pH in a range of 2-7 (or more particularly, a pH of about 2, 3, 4, 5, 6, or 7, or a pH in a range of 3-7, 4-7, 5-7, 2-6, 2-5, 2-4, 3-5, 3-6, or 4-6) before the aqueous solution makes contact with the hydrophobic solution. The buffer may be, for example, a lactate buffer, (2-(N-morpholino) ethanesulfonic acid) (MES) buffer, (4-(2-hydroxyethyl)-1-piperazineethanesulfonic acid) (HEPES) buffer, (3-(N-morpholino) propanesulfonic acid) (MOPS) buffer, or piperazine-N,N′-bis(2-ethanesulfonic acid (PIPES), all of which are well known in the art.

In separate or further embodiments, the aqueous solution further contains an electrolyte salt. The electrolyte salt is typically not a base, such as hydroxide. The electrolyte salt may be, for example, a substantially water-soluble inorganic salt, such as NaCl, KCl, NaBr, KBr, NH₄Cl, NaNO₃, KNO₃, (NH₄)₂SO₄, CH₃COO⁻Na⁺, MgCl₂, and MgBr₂. The ionic strength of the aqueous solution is typically precisely, about, or at least 0.1, 0.2, 0.3, 0.4, or 0.5 M. The aqueous solution typically does not include an acid.

The extraction process is capable of achieving a distribution coefficient (D), which may also herein be referred to as an extraction affinity, of at least 1 for one or more the lanthanide elements, wherein D is the concentration ratio of the rare earth element in the organic phase divided by its concentration in the aqueous phase. In some embodiments, a D value of greater than 1 is achieved, such as a D value of at least or above 2, 5, 10, 20, 50, 100, 150, 200, 250, 500, or 1000. The selectivity of the process can be characterized by the separation factor (SF), wherein SF is calculated as the ratio of D for two different ions, such as any two of the ions disclosed above, such as selectivity of an earlier lanthanide (e.g., Pr) relative to an later lanthanide (e.g., Eu), in which particular case SF=D_{Pr}/D_{Eu}. Selectivity is generally evident in an SF value greater than 1. In some embodiments, an SF value of at least or greater than 2, 3, 4, 5, 10, 20, 50, 100, 150, 200, 250, 500, or 1000 is achieved.

The method can separate lanthanide elements (denoted as Ln¹ and Ln² for lighter and heavier elements, respectively) with an Ln¹/Ln² selectivity of at least or greater than 2, 3, 4, 5, 6, 7, 8, 9, 10, 20, 30, 40, 50, 60, 70, 80, 90, or 100. Any lanthanide pair may be separated according to any of the foregoing selectivities. The lanthanide pair(s) may be, for example, La/Ce, La/Nd, La/Sm, La/Lu, Ce/Pr, Nd/Sm, Nd/Eu, Nd/Gd, Nd/Dy, Sm/Dy, Sm/Eu, Sm/Gd, Eu/Tb, Eu/Dy, and Eu/Lu. Moreover, one or more of the lanthanide elements may be radioisotopes, such as those useful in medical imaging, diagnostics, or therapy. Some examples of lanthanide radioisotopes include Tb-149, Lu-177, 153-Sm, 153-Gd, 141-Ce, and 166-Ho.

In some embodiments, the extraction method described above further includes a lanthanide (or rare earth metal) removal step from the hydrophobic solution, aqueous solution, or both. To remove one or more lanthanides from the hydrophobic solution, the hydrophobic solution may be contacted with an aqueous stripping solution containing at least one inorganic acid, such as any of the inorganic acids described above, wherein the inorganic acid is typically present in a concentration of no more than 4 M (e.g., 0.5, 1, 2, or 3 M). Generally, the concentration of inorganic acid in the aqueous stripping solution is at least 0.5 M less (or at least 1 M, 1.5 M, 2 M, 3 M, or 4 M less) than the concentration of inorganic acid in the aqueous source solution in step (i). To remove one or more lanthanides from the aqueous solution, ammonium bicarbonate may be added to the aqueous solution to induce precipitation of the one or more lanthanides or rare earth elements.

Examples have been set forth below for the purpose of illustration and to describe certain specific embodiments of the invention. However, the scope of this invention is not to be in any way limited by the examples set forth herein.

### Examples

### Overview

The present work prepared a flexible amphiphilic ligand, coined “octadecyl acyclopa” (ODA), that features a diaza polyethylene glycol podand appended with two chelating picolinic acid pendants and two octadecyl endcap groups. The structure of ODA is as follows:

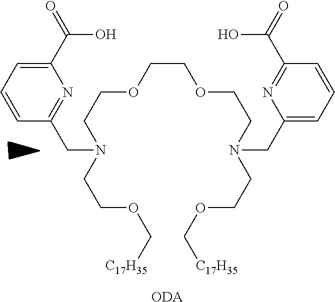

This molecule is an acyclic (linear) variation of the conventional cyclic macropa chelator, i.e., by scission of a single bond of the cyclic version. As further discussed below, extraction experiments show that despite the loss of conformational rigidity afforded by the macrocyclic backbone of macropa, the linear ODA extractant is capable of preferentially stabilizing lighter Ln³⁺ ions versus the heavier species, in contrast to the conventional view of lanthanide coordination chemistry. Surface-specific vibrational sum frequency generation (SFG) spectroscopy shows that ODA arranges at organic/aqueous interfaces to form pseudocyclic structures, analogous to macropa, that are transported to the organic phase on extraction. The interfacial structures are found to be independent of the specific Ln³⁺ ions, indicating that the stabilization is realized thermodynamically vs. kinetically. This finding is supported by ultrafast two-dimensional infrared spectroscopy (2D IR), density functional theory (DFT) calculations, and ab initio molecular dynamics (AIMD) simulations that connect conformational dynamics, binding energies, and strain to selectivity. These results show that the reverse-size selectivity arises from the inability of the flexible ligand to adopt the optimal geometry needed to effectively coordinate the heavier, smaller, lanthanides, despite the ability to form individually stronger bonding interactions. It is herein also shown that for molecules of this size, energy dissipation to outer-sphere constituents plays a secondary role to the number and strength of inner-sphere coordination interactions. These results point to a new paradigm in ligand design where conformational flexibility is embraced with an eye toward engineering collective interactions in Ln³⁺ coordination.

The novel extractant octadecyl acyclopa (ODA) was prepared over five steps, as shown in the following scheme:

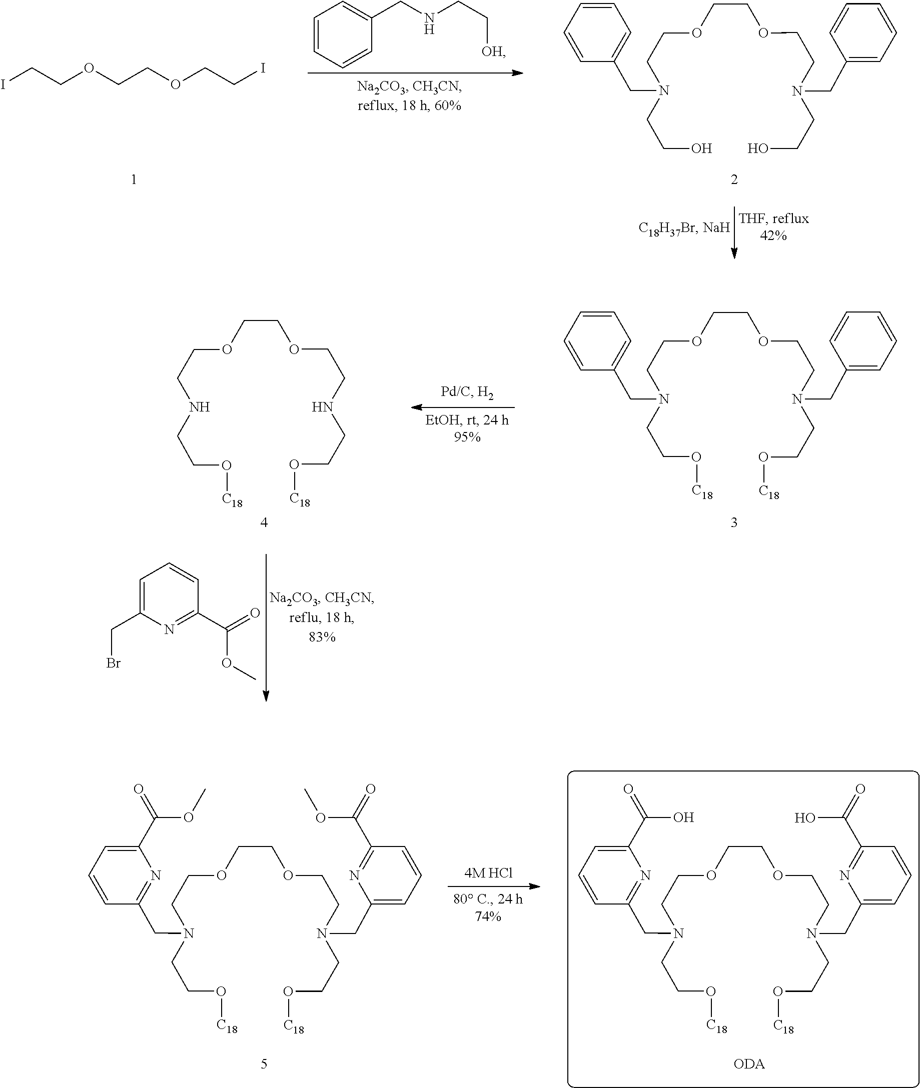

In the first step, 1,2-bis(2-iodoethoxy) ethane (1) was treated with two equiv N-benzylethanolamine in a straightforward N-alkylation reaction to form the benzyl protected core acyclic scaffold, 2. Next, the hydroxy groups of compound 2 were deprotonated using NaH in THF, and lipophilic C18 ‘tails’ were installed via O-alkylation with 1-bromooctadecane to afford compound 3. The benzyl groups of 3 were removed with H₂ over Pd/C catalyst, forming 4, and then the amino nitrogen atoms of 4 were alkylated with methyl-(6-bromomethyl) picolinate to install the picolinic acid pendent arms. Finally, acid hydrolysis of the picolinate esters yielded the pure ligand as a white solid. The final product and the intermediates were analyzed by nuclear magnetic resonance (NMR) spectroscopy, high-resolution mass spectrometry (HRMS), and analytical high performance liquid chromatography (HPLC) techniques to ensure the identity and purity of ODA. Notably, although a relatively low global yield of 16% was obtained for ODA, the synthesis did not require the demanding high dilution conditions and templating typically needed for forming macrocycles. It also allows for the modular installation of a range of pendant arms and tails in future design iterations. Yields may be improved henceforth by omitting the benzyl protection step and forming the acyclic diaza-crown-6 scaffold directly.

### Synthesis and Characterization of Octadecyl Acyclopa (ODA)

### (i) Synthesis of 3, 12-dibenzyl-6,9-dioxa-3,12-diazatetradecane-1,14-diol (2)

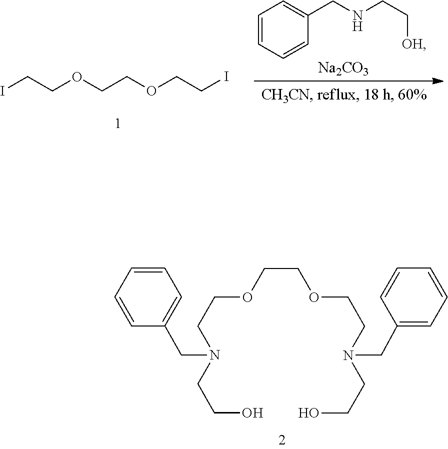

Compound 2 was synthesized according to a previously reported method with slight modification. Briefly, in a round-bottom flask, compound 1 (10.0 g, 27.03 mmol) was dissolved in CH₃CN (100 mL). Then, N-benzylethanolamine (8.257 g, 54.61 mmol) and sodium carbonate (17.191 g, 162.20 mmol) were added. The mixture was heated to reflux and stirred for 18 h. The pale-yellow suspension was then filtered to remove excess solids. The filtrate was concentrated at 50° C. on the rotary evaporator to a yellow oil. The yellow oil was dissolved in 100 mL of ethyl acetate and washed with H₂O (50 mL×3). The organic layer was dried over sodium sulfate and concentrated at 50° C. on the rotary evaporator to give a crude yellow oil. The crude material was then purified on normal phase silica using flash column chromatography (100% CH₂Cl₂→90% CH₂Cl₂: 10% CH₃OH), yielding the title compound 2 as a slightly yellow oil (6.747 g, 60%). 1H NMR (400 MHZ, CDCl₃) δ 7.35-7.27 (m, 8H), 7.23 (td, J=5.7, 3.0 Hz, 2H), 3.70 (s, 4H), 3.59-3.49 (m, 12H), 3.28 (s, 2H), 2.72 (m, 8H). ¹³C {1H} NMR (101 MHZ, CDCl₃) δ 139.3, 129.0, 128.4, 127.2, 70.4, 69.9, 59.6, 59.2, 56.1, 53.1.

### (ii) Synthesis of diethyl 2,2′-(ethane-1,2-diylbis(oxy))bis(N-benzyl-N-(2 (octadecyloxy)ethyl) ethan-1-amine) (3)

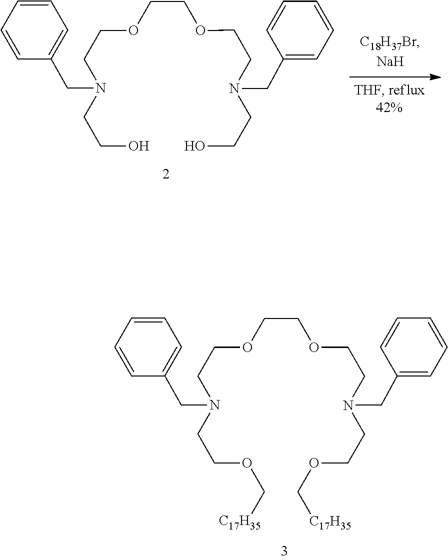

In a 2-neck round-bottom flask under Ar, sodium hydride (NaH, 5.473 g, 136.84 mmol) was suspended in dry tetrahydrofuran (THF, 50 mL) at 0° C. Then, 2 (9.501 g, 22.81 mmol) was dissolved in 100 mL of dry THF and added slowly to the suspension of NaH over the course of 1 h. The mixture was removed from the ice bath and heated at reflux for 2 h. The mixture was then cooled to room temperature and 1-bromooctadecane (18.69 mL, 54.73 mmol) in 54 mL of dry THF was added to the reaction mixture. The mixture was heated to reflux for 18 h and then cooled to room temperature. The reaction was quenched via careful addition of H₂O (200 mL), and the mixture was extracted with CH₂Cl₂ (100 mL×3). The organic phase was dried over sodium sulfate and concentrated at 50° C. on a rotary evaporator to a colorless oil. The crude oil was further purified using flash column chromatography by normal phase silica (100% CH₂Cl₂→70% CH₂Cl₂: 30% CH₃OH) to obtain product 3 as a colorless oil (8.835 g, 42%). 1H NMR (400 MHZ, CDCl₃) δ=7.30 (q, J=6.9 Hz, 8H), 7.22 (d, J=7.6 Hz, 2H), 3.69 (s, 4H), 3.58-3.51 (m, 8H), 3.48 (t, J=6.6 Hz, 4H), 3.36 (t, J=6.8 Hz, 4H), 2.73 (q, J=6.8 Hz, 8H), 1.60-1.47 (m, 4H), 1.25 (s, 59H), 0.88 (t, J=6.2 Hz, 6H). ¹³C {1H} NMR (101 MHZ, CDCl₃) δ 139.9, 128.9, 128.2, 126.9, 77.5, 76.8, 71.4, 70.5, 70.1, 69.6, 59.9, 54.0, 53.9, 32.1, 29.9, 29.9, 29.8, 29.8, 29.7, 29.5, 26.3, 22.8, 14.3. ESI-MS: Found: m/z 921.8374. Calculated for [C₆₀H₁₀₈N₂O₄+H]⁺: m/z 921.8387.

### (iii) Synthesis of 2,2′-(ethane-1,2-diylbis(oxy))bis(N-(2-(octadecyloxy)ethyl) ethan-1-amine) (4)

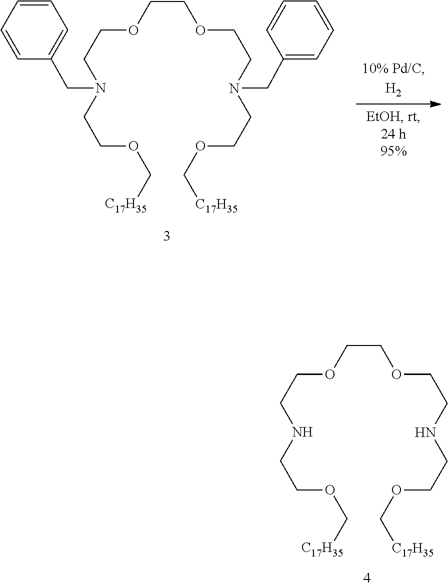

In a 250 mL round-bottom flask under Ar, compound 3 (3.517 g, 3.79 mmol) was dissolved in 20 mL CH₂Cl₂. Then, 10% Pd/C, type 487 (0.606 g) was added under Ar. The mixture was further diluted with ethanol (60 mL). The flask was then filled with H₂ gas using two balloons, and the mixture was stirred at room temperature for 18 h. Once the reaction was confirmed to be complete by thin layer chromatography, the suspension was filtered through a nylon membrane, and the filtered solid was further washed with excess CH₂Cl₂ (100 mL). The filtrate was concentrated at 50° C. on the rotary evaporator to give a white solid. The white solid was further dried in vacuo to yield pure product 4 (2.683 g, 95% yield). 1H NMR (400 MHZ, CDCl₃) δ 3.62-3.54 (m, 8H), 3.50 (t, J=5.4 Hz, 4H), 3.40 (t, J=6.8 Hz, 4H), 2.79 (m, J=8.4, 5.3 Hz, 8H), 1.54 (p, J=6.7 Hz, 4H), 1.23 (s, 62H), 0.90-0.82 (m, 6H). ¹³C {1H} NMR (101 MHZ, CDCl₃) δ 77.5, 76.8, 71.4, 70.9, 70.5, 70.3, 49.6, 49.5, 32.0, 29.8, 29.8, 29.8, 29.8, 29.8, 29.6, 29.5, 26.3, 22.8, 14.2. ESI-MS: Found: m/z 741.7464. Calculated for [C₄₆H₉₆N₂O₄+H]⁺: m/z 741.7443.

### (iv) Synthesis of dimethyl 6,6′-(2, 11-bis(2-(octadecyloxy)ethyl)-5,8-dioxa-2,11-diazadodecane-1,12-diyl)dipicolinate (5)

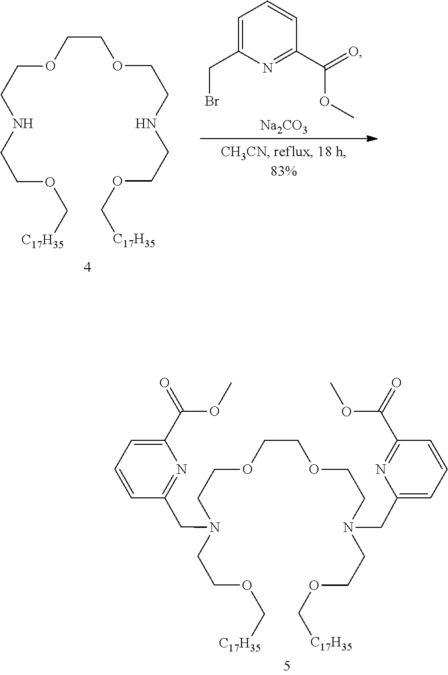

In a 1 L round-bottom flask, 4 (2.683 g, 3.62 mmol) was dissolved in 100 mL of dry CH₃CN. Then, Na₂CO₃ (1.917 g, 18.09 mmol) was added. The mixture was heated to reflux. After 2 h, methyl 6-(bromomethyl) picolinate (1.665 g, 7.24 mmol) was added. The suspension was stirred at reflux for 16 h and then filtered. The filtrate was concentrated at 50° C. on a rotary evaporator to obtain a slightly yellow oil. The crude oil was further purified using flash column chromatography with normal phase silica (100% CH₂Cl₂→90% CH₂Cl₂: 10% CH₃OH) to yield the product 5 as a waxy off-white solid (3.031 g, 83%). 1H NMR (400 MHZ, CDCl₃) δ 7.97 (d, J=6.4 Hz, 2H), 7.84 (d, J=7.8 Hz, 2H), 7.77 (t, J=7.7 Hz, 2H), 3.98 (s, 10H), 3.54 (t, J=6.1 Hz, 4H), 3.52-3.44 (m, 8H), 3.33 (t, J=6.7 Hz, 4H), 2.79 (m, 8H), 1.50 (p, J=6.8 Hz, 4H), 1.25 (s, 60H), 0.91-0.83 (m, 6H). ¹³C {1H} NMR (101 MHz, CDCl₃) δ 166.1, 161.9, 147.2, 137.3, 126.2, 123.6, 71.5, 70.5, 69.9, 69.4, 61.6, 54.7, 54.5, 53.0, 32.1, 29.9, 29.9, 29.8, 29.8, 29.7, 29.5, 26.3, 22.8, 14.3. ESI-MS: Found: m/z 1039.8423. Calculated for [C₆₂H₁₁₀N₄O₈+H]⁺: m/z 1039.8396.

### (v) Synthesis of Octadecyl-Acyclopa (ODA)

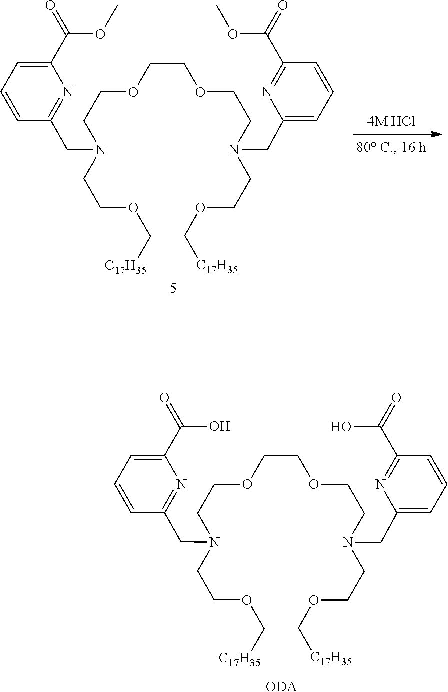

To a 50 mL round-bottom flask charged with 5 (2.012 g, 1.98 mmol) was added HCl (4 M, 45 mL). The mixture was then heated to 80° C. for 14 h. An off-white suspension was formed, which was cooled to room temperature and then filtered through a nylon membrane. The residue was washed with ice-cold methanol and then redissolved in CHCl₃ (10 mL). The product was precipitated by using excess diethyl ether (100 mL) at −25° C. The precipitate was filtered through a nylon membrane. The residue was dried in vacuo to yield ODA as an off-white powder (1.443 g, 74%). 1H NMR (400 MHZ, C₂D₂Cl₄) δ 8.17 (d, J=7.7 Hz, 2H), 7.97 (t, J=7.8 Hz, 2H), 7.66 (d, J=7.7 Hz, 2H), 4.86 (s, 4H), 3.99 (d, J=42.7 Hz, 8H), 3.54 (s, 12H), 3.39 (t, J=6.7 Hz, 4H), 1.50 (t, J=6.5 Hz, 4H), 1.24 (s, 60H), 0.94-0.82 (m, 6H). ¹³C {1H} NMR (101 MHz, CDCl₃) δ 165.6, 150.4, 147.4, 139.4, 126.9, 124.3, 71.7, 70.4, 65.7, 65.3, 58.2, 55.0, 54.9, 31.9, 29.7, 29.7, 29.5, 29.4, 26.1, 22.7, 14.2. ESI-MS: Found: m/z 1011.8071. Calculated for [C₆₀H₁₀₆N₄O₈+H]⁺: m/z 1011.8089. Elemental Analysis, Found: C=64.70, H=10.03, N=5.01, C_{1=6.31}. Calculated for C₅₈H₁₀₂N₄O₈·2HCl·2H₂O: C=64.32, H=10.08, N=5.00, C_{1=6.33.}

### Ln³⁺ Distribution Ratios, Conditional Extraction Constants, and Separation Factors

Organic phase preparation. Organic stock solutions of ODA were prepared at various concentrations (0.1-12 mM) in volumetric flasks using 1,1,2,2-tetrachloroethane (TCE).

Aqueous phase preparation. Lactate buffer (50 mM) containing NaCl (0.2 M) was prepared at pH 4 using lactic acid (1.01 N) and NaCl (puriss grade). Different metal stock solutions containing (La, Ce), (Pr, Nd), (Sm, Eu), (Gd, Tb, Dy), or (Ho, Er, Tm) were prepared by combining aliquots of ICP standards of the individual elements and diluting to the appropriate volume with MQ H₂O to arrive at a final concentration of 4 mM for each Ln³⁺ in solution.

Liquid-liquid extraction experiments. ODA stock solutions were first pre-conditioned with lactate buffer. In this pre-conditioning process, equal volumes of ODA in TCE and buffer were contacted for 2 minutes. The two phases were separated, and the aqueous phase was discarded. The organic phase was contacted again with fresh buffer. This process was repeated multiple times until the pH of the contacted buffer was the same as that of the uncontacted buffer. The organic phase containing ODA was then separated and used for extraction experiments. All experiments were conducted at 25±1° C. Samples were prepared by combining lactate buffer (0.99 mL), Ln sub-group stock solutions (0.01 mL, 0.04 mM final concentration of each metal), and ODA (0.1-12 mM, 1 mL) in 5 mL screw-cap polypropylene Eppendorf tubes. The samples were contacted for 5 min-18 h by end-over-end rotation at 40 RPM. Because of the large differences in stability between the Ln³⁺ complexes of ODA as a function of metal-ion size, experiments were unable to obtain measurable distribution ratios using a single concentration of ODA. Therefore, the concentration of ODA was varied such that the distribution of each Ln³⁺ could be accurately measured in both phases. Control samples were also prepared in which ODA ligand was omitted. The concentration of Ln in the aqueous phases of these control samples were found to match the theoretical value, confirming that no Ln partitioning occurs in the absence of ligand. After the designated time points, the samples were centrifuged at 4000 RPM for 10 min, and the aqueous phase was carefully transferred to a 1.0 mL Eppendorf vial until it could be analyzed by ICP-MS (see below). After an aliquot of each sample was removed for ICP-MS analysis, the pH of the remaining aqueous phase was measured and recorded for each sample. The equilibrium pH values for samples initially prepared at pH 4 were 3.88-3.95.

ICP-MS measurements. All samples and calibration standards for ICP-MS were prepared in 2% HNO₃ containing¹¹⁵ In as an internal standard. Specifically, ICP samples were prepared by diluting the aqueous phase from extraction experiments (100 μL) with HNO₃ (9.900 mL) to give a dilution factor of 100. The calibration standards and diluents were prepared using a 99:1 ratio of 2% HNO₃ and buffer solution to match the ICP sample composition as closely as possible, thereby helping to minimize matrix effects. The following isotopes were monitored: ¹³⁹La, ¹⁴⁰Ce, ¹⁴¹Pr, ¹⁴³Nd, ¹⁴⁶Nd, ¹⁴⁷Sm, ¹⁴⁹Sm, ¹⁵³Eu, ¹⁵⁵Gd, ¹⁵⁷Gd, ¹⁵⁸Gd, ¹⁵⁹Tb, ¹⁶²Dy, ¹⁶³Dy, ¹⁶⁴Dy, ¹⁶⁵Ho, ¹⁶⁶Er, ¹⁶⁷Er, ¹⁶⁹Tm, ¹⁷²Yb, ¹⁷⁴Yb, and ¹⁷⁵Lu. Blanks and calibration standards were re-analyzed periodically to check for sample carryover and loss of calibration, respectively; neither issue was observed during these experiments.

Data Treatment. Raw counts were normalized to the internal 115 In standard, of which the intensity remained between 90% and 100% of its calibrated values throughout the experiments. Calibration curves comprising data from the blank and eight Ln concentrations (0.33-100 ppb) were linear throughout the concentration range, with correlation coefficients >0.999. The concentration of Ln remaining in the aqueous phase of each sample at equilibrium (t=18 h) was calculated from the isotope-specific calibration curves. The concentration of Ln in the organic phase of each sample was calculated from the difference between the initial and equilibrium concentrations of Ln³⁺ in the aqueous phase. The distribution ratio of each Ln³⁺ in a sample, D_{M}, was determined by dividing [M]org by [M]aq. Using the previously established extraction equilibrium equation (Eq S1), the D_{M} values for the Ln/ODA system were then normalized to account for the different concentrations of ODA used in the extraction experiments according to Eq S2.2. The overbars indicate organic-phase species.

$${M}^{3} + {H}_{n} \overline{( ODA )} ⇌ \overline{M ( ODA )} + {nH}^{+} \qquad ( Eq S1 )$$

$${K}_{ex} = {D}_{M} \frac{{[ {H}^{+} ]}^{n}}{[ \overline{( ODA )} ]} \qquad ( Eq S2 )$$

Kₑₓ is the conditional extraction constant, and [H⁺] is calculated from the measured pH of each sample at equilibrium. The n term was taken as a constant across all samples (lanthanides) and set equal to 1 for the purposes of comparing relative Kₑₓ values. The equilibrium concentration of ODA in the organic phase ([(O̅D̅A̅)]) for each sample was calculated in the same manner as described previously for HDEHP (Thiele, N. A.; et al. Inorg. Chem. 2020, 59, 16522-16530. DOI: 10.1021/acs.inorgchem.0c02413). To reiterate, the total concentration of Ln in the organic phase of each sample was determined from the data obtained from ICP-MS. This value was then subtracted from the initial concentration of ([(O̅D̅A̅)]) added to each sample. This calculation is critical to obtaining accurate Kₑₓ values when only small amounts of ODA are used in extractions (e.g., 10-fold excess ODA with respect to total metal ion concentration), for which the initial and equilibrium concentrations of ODA cannot be considered equal. Kₑₓ values were averaged across the different isotopes monitored for different lanthanides.

Sample preparation for Sum Frequency Generation (SFG) Vibrational Spectroscopy: A stock solution of 23.13 mM of ODA was first prepared in ethanol. The stock was subsequently diluted 25-fold using n-decane to a final concentration of ˜1 mM. Individual stock solutions of La, Gd and Lu were prepared in 0.1 M HCl from their chloride salts, and final concentrations were determined using ICP-MS (La=0.954 M±0.003 M, Gd=0.961 M±0.001 M, Lu=0.943 M±0.008 M). Individual metal solutions were diluted to a concentration ˜64 μM in 50 mM lactate buffer (pH 4) containing 0.2 M NaCl. These aqueous phases were then contacted with the organic phase containing ODA (vide infra).

Sample preparation for Two-Dimensional Infrared Spectroscopy (2D IR): 2-(N-morpholino) ethanesulfonic acid (MES) buffer (50 mM) containing NaCl (0.2 M) was prepared at pH 6.5 using MES hydrate (MW=213 g/mol) and NaCl (puriss grade). To prepare the samples for 2D IR, first a stock solution of ODA (1 mM) was prepared by dissolving 0.220 g of ODA (0.199 mmol) into 200 mL of TCE. Then, the solution was pre-conditioned using MES buffer (pH=6.5). Separately, La, Gd and Lu solutions were each prepared in MES buffer (50 mL each, 1 mM). Then, extraction experiments were carried out by combining 50 mL of ODA stock with 50 mL of La/Gd/Lu solution in buffer in 250 mL Erlenmeyer flasks. They were stirred vigorously at 25° C. for 18 h. Then, the aqueous phase was separated following centrifugation. The aqueous phase was analyzed using ICP-MS to obtain the percentage loading of the Ln³⁺ ions by ODA. The organic phases were then concentrated to ˜1 mL (˜50 mM Ln³⁺ and ODA) via distillation and used directly for 2D IR measurements.

### Results and Discussion

### Lanthanide Solvent Extraction Studies Reveal ODA's Reverse-Size Selectivity.

To evaluate the Ln selectivity preferences of ODA as a conformationally flexible ligand, extraction experiments were carried out on a mixture of Ln³⁺ ions in a biphasic system comprising pH 4 lactate buffer and 1,1,2,2-tetrachloroethane (TCE). Once equilibrium was reached, the concentration of each Ln³⁺ ion remaining in the aqueous phase was measured using inductively coupled plasma-mass spectrometry (ICP-MS). The Ln³⁺ distribution ratio (D_{M}), a measure of the partitioning of ions between the organic and aqueous phases, was subsequently obtained by mass balance. Studies were limited to La—Tm due to the extremely low extraction of the heavier Ln³⁺ ions into the organic phase, which precluded the accurate determination of Yb and Lu distribution between the phases under the conditions used.

With D_{M} values in hand, slope analysis was performed by plotting log D_{M} vs. log ODA concentration (0.5-12 mM) to assess the stoichiometry of extracted complexes. These plots yielded a linear trend with a slope of ˜1 across the series, suggesting that ODA forms mononuclear complexes with both large and small Ln³⁺ ions. To account for the variation in ODA concentration between samples, the D_{M} values were normalized to determine conditional extraction constants (Kₑₓ), which can be directly compared across the series.

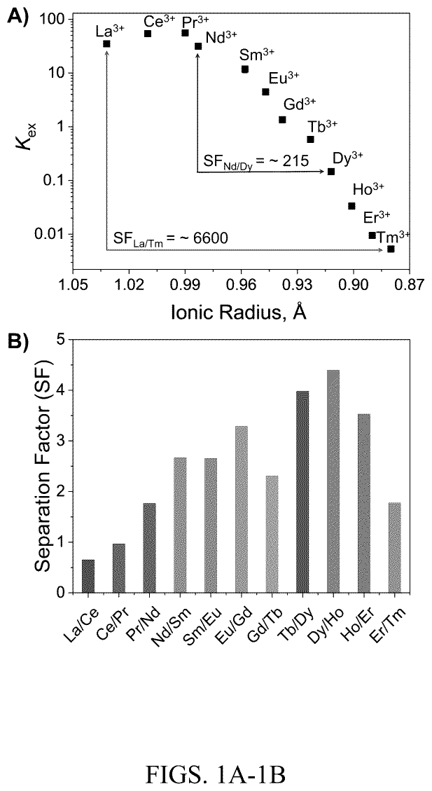

The results in FIG. 1A show that ODA facilitates the preferential partitioning of light Ln³⁺ to the organic phase from the aqueous phase, and therefore retains the reverse-size-selectivity pattern of the parent macrocycle. The highest Kₑₓ values for extraction by ODA are observed for La³⁺, Ce³⁺, Pr³⁺, and Nd³⁺, with peak selectivity at Pr³⁺. With decreasing ionic radius from these light Ln³⁺ ions to Tm³⁺, Kₑₓ values decrease dramatically, indicating that Ln-ODA complexes are progressively destabilized across the series. Notably, this general selectivity pattern mirrors that of macropa in aqueous solution based on experimentally determined stability constants (log KML values). From the ratio of Kₑₓ values, separation factors (SF=Kₑₓ (Ln¹)/Kₑₓ (Ln²)) of adjacent pairs of Ln³⁺ ions were calculated, with the results shown in FIG. 1B. SFs ranged from 0.7 for the La/Ce pair to 4.4 for the Dy/Ho pair and tended to be greater for the mid-to-late Ln. Thus, ODA more effectively discriminates between the later Ln than between the earlier Ln, consistent with the steep decline in Kₑₓ values across the series after Nd³⁺. This decline gives rise to a SF of 6600 for the largest/smallest Ln pair studied, La³⁺ and Tm³⁺, which is among the highest trans-lanthanide separation factors reported to date for a reverse-size selective extractant. A high SF of 215 was also achieved for the Nd/Dy pair, two Ln³⁺ ions whose separation is crucial in the context of recovering Ln from secondary sources such as magnet waste. Collectively, these results reveal ODA's ability as a new extractant for unconventional Ln separations.

### ODA Forms a Pseudocyclic Structure at the Liquid-Liquid Interface.

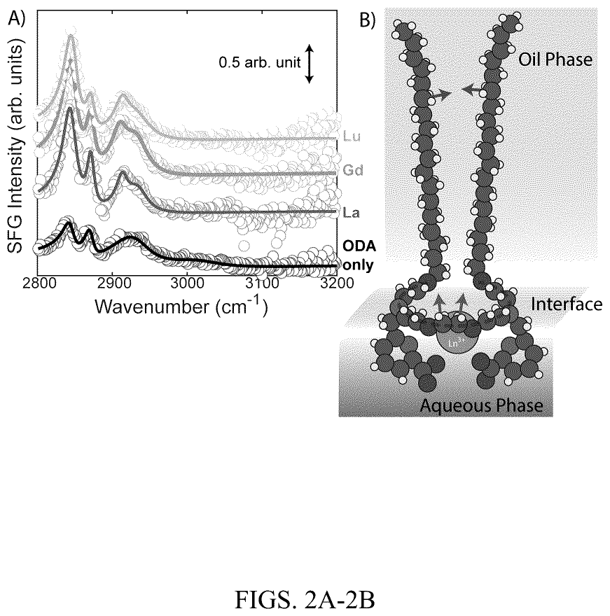

Having established the reverse-size binding preferences of ODA, next experiments sought to understand the molecular origins of its selectivity. As a first step, vibrational sum frequency generation (SFG) spectroscopy was employed. As well known, SFG is a nonlinear surface-specific spectroscopy capable of structurally characterizing species at buried interfaces. Since extracted species are formed at and traverse the organic/aqueous interface in a separation, the molecular assemblies located there at equilibrium inform on the structural motifs adopted during solvent extraction. SFG spectra of ODA at an interface of n-hexane and pH 4 lactate buffer were acquired in the absence and presence of La³⁺, Gd³⁺ and Lu³⁺ and are plotted in FIG. 2A in the SSP polarization combination, which preferentially probes symmetric stretches pointed out of the interfacial plane. La, Gd, and Lu were studied as representative examples of large, medium, and small Ln³⁺ ions. The signal at ˜2850 cm⁺¹ corresponds to methylene symmetric stretches (—CH₂-ss) on the diaza crown backbone and the alkyl tails (vide infra), whereas the peak near 2875 cm⁺¹ corresponds to methyl symmetric (—CH₃-ss) stretches from the alkyl tails. The overlapping features at ˜2926 cm⁺¹ and 2946 cm⁺¹ are assigned to Fermi resonances with unresolved and out of phase contributions from methyl and methylene asymmetric (-as) stretches. The contribution from polarized bulk water molecules in these experiments is small (—OH stretches, >3000 cm⁺¹, FIG. 2A), possibly because the acidic picolinate groups are not completely ionized at pH 4. More likely, however, the small water contributions can be explained by the fact that a buffered aqueous phase with moderate ionic strength (0.2 M NaCl) was used in experiments, which screens the surface potential.

From these SFG spectra, a structural picture of ODA at the interface begins to emerge, as sketched in FIG. 2B. As well known, the methylene stretches from hydrophobic tails at a well-packed surface tend to be small for extractants and other amphiphiles due to azimuthal averaging in the inter-facial plane. This finding is supported by orientational analysis that shows the average —CH₃ tilt angles do not vary in the presence of Ln³⁺ ions, which is consistent with the localization of the long tails in the oil phase, rendering them conformationally independent from groups pinned to the interface. As such, the large intensity of the —CH₂-ss band is primarily attributed to the methylene groups of the acyclic diaza crown backbone of ODA. Furthermore, this band intensity increases in the presence of Ln³⁺ ions, thus indicating structural rearrangements induced by Ln binding. Thus, it may be proposed that the crown backbone of ODA arranges flat at the interface in a pseudocyclic configuration, thereby allowing the hydrophilic ether O-atoms to interact with the aqueous phase while enabling the hydrophobic methylene groups to point towards the oil phase. This configuration would optimally place the picolinate groups into the aqueous phase. Notably, the SFG spectra do not vary substantially between La³⁺, Gd³⁺, and Lu³⁺. Therefore, although there is a notable Ln-driven change in the pseudocyclic structure of ODA upon binding, the structures formed are largely independent of the identity of the Ln³⁺ that is coordinated. Taken together, these observations indicate that the interfacial arrangement is important to enable extraction, but in these systems at steady state, the surface does not define the selectivity.

Structural Dynamics of Extracted ODA Complexes Vary with Ion Size.

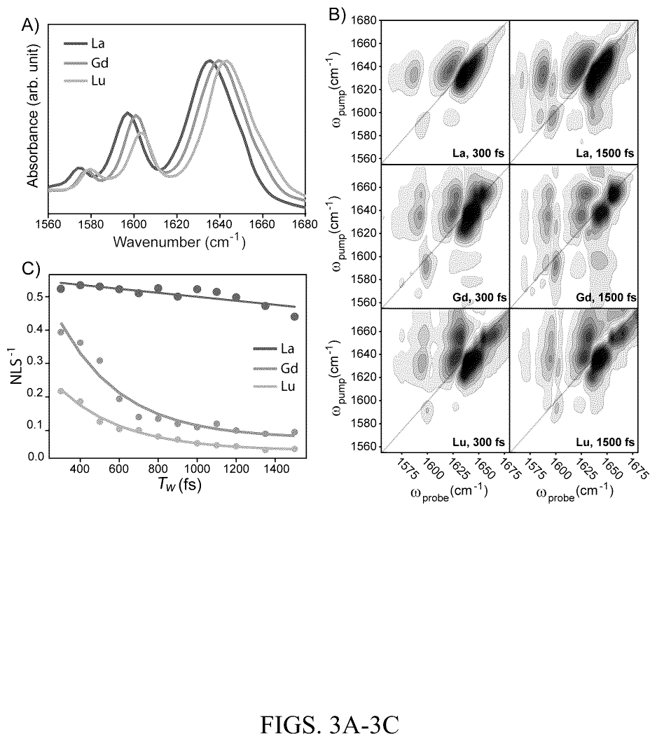

Next experiments were directed toward probing the nature of the Ln-ODA complexes extracted into the bulk phase, away from the interface, using Fourier transform infrared (FTIR) and 2D IR spectroscopies. These studies were performed on ODA species in TCE after loading the ligand with La³⁺, Gd³⁺, or Lu³⁺ from pH 6.5 MES buffer. FIG. 3A shows the FTIR spectra of the three Ln³⁺ complexes, focused on the region from 1560 to 1680 cm⁺¹. Here, two major peaks, centered at ˜1600 cm⁺¹ and ˜1640 cm⁺¹, are clearly resolved. These peaks are assigned to the C—C and C—N stretching modes of the pyridine rings and asymmetric stretches of the pyridyl carboxylate groups, confirmed by DFT calculations below. Both peaks show characteristic shifts in frequency based on the identity of the Ln³⁺ ion in solution binding to the carboxylates. The blue shift observed with increasing atomic number is due to the higher charge densities of heavier Ln³⁺ ions that result in individually stronger bonds. Taken at face value, the FTIR data predict conventional selectivity of ODA for Ln³⁺ ions, and yet, solvent extraction experiments show the opposite trend. The question may then be posed as to how ODA can form stronger Ln-ligand bonds with small Ln³⁺, but yet these complexes do not excel thermodynamically in extractions.

To address this question, next experiments studied the conformational dynamics of the Ln³⁺-ODA complexes using femtosecond 2D IR measurements, which are plotted in FIG. 3B. 2D IR measurements are uniquely capable of separating overlapping spectral responses from isomers, resolving chemical exchange, and mapping ultrafast structural dynamics in complex chemical systems. For example, the 2D IR spectrum of La³⁺-loaded ODA exhibits two bands along the diagonal at 1600 cm⁺¹ and 1640 cm⁺¹, in agreement with the FTIR spectrum. Similar spectral features were observed for Gd— and Lu-ODA complexes; however, two bands near 1640 cm⁺¹ are resolved in the 2D IR spectra. This splitting suggests the presence of different conformers (vide infra), but the lack of cross peaks between them indicates that they do not interconvert on the ps timescale studied here.

The time evolution of a 2D IR spectral line shape reports directly on conformational dynamics by mapping a frequency-frequency correlation function. Considering the 1640 cm⁺¹ bands, it was herein found that for Gd and Lu complexes, the 2D IR line shapes are rounder, whereas the La band is more elongated along the diagonal. The increasing symmetry of the 2D line shape indicates a loss of memory of the initial distribution of vibrational states that were created by the pump pulses at t=0 fs. This memory is lost through interactions with local chemical environments as measured by a time-delayed probe pulse after a waiting time, Tw, a process known as spectral diffusion. To quantify these conformational fluctuations and dynamics, experiments measures the inverse nodal line slopes (NLS⁻¹) that appear between positive and negative signed features in the 2D IR spectra and plot them vs. T_{w} in FIG. 3C. For Gd³⁺ and Lu³⁺ loaded complexes, the NLS⁻¹ decay exponentially with increasing T_{w}, with time constants of 323±62 fs and 333±33 fs, respectively. However, for the La³⁺ loaded complex, the NLS⁻¹ remains almost constant over the experimental window. This lack of change means that the ligand conformation in the La-ODA complex is relatively rigid and invariant over 1.5 ps. By contrast, Gd-ODA and Lu-ODA undergo ultrafast conformational fluctuations around the Ln³⁺ binding site. This finding can be interpreted as ions ‘rattling’ between binding sites, unable to sate all of the would-be coordinating atoms at once due to the small size of heavier Ln³⁺ ions.

Cumulative Stabilization through Backbone Interactions Leads to Selectivity.

Lastly, to lend support to the conclusions drawn from SFG, FTIR, and 2D IR data, DFT and AIMD simulations probing the structural and energetic differences between the extracted metal-ligand assemblies were carried out. To balance accuracy and computational cost, these studies used a truncated version of ODA, referred to as ODAMe, in which the octadecyl tails are replaced with methyl substituents. Starting from the optimized structure of macropa published previously (Roca-Sabio et al., J. Am. Chem. Soc. 2009, 131, 3331-3341. https://doi.org/10.1021/ja808534w), the geometries of 32 distinct conformers of ODAMe complexed with La³⁺, Gd³⁺, or Lu³⁺ were optimized at the M06/LC (Ln)/def2TZVPP level of theory (Y. Zhao et al., Theor. Chem. Acc. 2008, 120, 215-241. https://doi.org/10.1007/s00214-007-0310-x). The conformational analysis reveals a general pseudocyclic arrangement for the core linear scaffold of ODAMe when complexing all three Ln³⁺, in agreement with SFG data. The lowest energy configuration for La³⁺ and Gd³⁺ complexes is A (82282), whereas the present calculations predict that the most stable configuration for Lu³⁺ is A (28222), albeit by only 0.11 kcal/mol. Upon closer inspection of the lowest energy conformers, however, a size-dependent trend can be discerned. Namely, the A (82282) conformation, lowest in energy for La³⁺, is progressively destabilized across the series by 1.28 kcal/mol. A more significant destabilization is also observed for the second lowest energy conformation of La-ODAMe, A (28828), which increases in energy by 4.5 kcal/mol from La³⁺ to Lu³⁺. Although this trend is not as pronounced as that observed for macropa, it nonetheless suggests a possible switch in the conformation of ODA when binding large versus small Ln³⁺ ions.

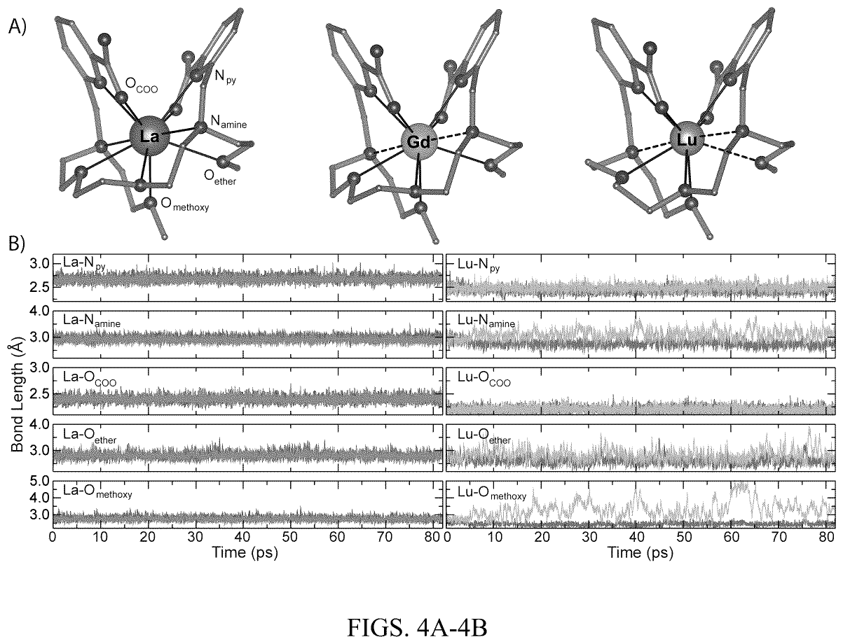

A comparison of interatomic distances also shows marked differences between the three complexes with respect to their bonding interactions. As the lanthanide series is traversed, interatomic distances between the metal center and donor atoms are expected to decrease due to the contracting ionic radius of the Ln³⁺ ion. This trend is observed for bonds arising from the picolinate N_{py} and O_{COO} donor atoms of the DFT-optimized ODAMe complexes, which decrease by ˜0.16 and 0.18 Å, respectively, from La³⁺ to Lu³⁺. By contrast, the interatomic distances between the Ln³⁺ ion and several O and N donors of the acyclic backbone are nearly invariant across the complexes, thus indicating a weakening of these bonds with decreasing ionic radius. These results suggest that ODAMe cannot effectively contort around the smaller ions, despite its apparent flexibility, thereby limiting interactions with all the coordinating groups on the ligand. FIG. 4A shows DFT-optimized structures of the ODAMe complexes with La³⁺, Gd³⁺, and Lu³⁺ ions. Solid lines represent the stronger Ln-O or Ln-N bonds in ODAMe complexes, whereas dashed lines represent the weaker Ln-O and Ln-N bonds in the Gd— and Lu-ODAMe complexes.

These results are reinforced by AIMD simulations, which measure bond distances vs. time for the La³⁺ and Lu³⁺ complexes of ODAMe, with the results shown in FIG. 4B. In agreement with configurationally optimized DFT calculations above, the bond lengths show marked differences between coordination sites on ODAMe but also report on fluctuations in those distances vs. time. Specifically, while the Ln-N_{py} and Ln-O_{COO} bond lengths in the Lu³⁺ complex follow the same trend as seen in the case of hydrated metal nitrate complexes (A. S. Ivanov et al., Eur. J. Inorg. Chem. 2016, 2016, 3474-3479. https://doi.org/10.1002/ejic.201600319), bonds formed between Nₐₘᵢₙₑ, Oₑₜₕₑᵣ, and O_{methoxy} donor atoms and the metal center fluctuated significantly over time. Consequently, these bonds are markedly elongated relative to the bond lengths expected based on ionic radius. This geometric and dynamic analysis suggests that the reverse-size selectivity arises because ODA cannot adjust its structure to effectively complex the smaller ions, thus highlighting a limited complementarity of the ligand to smaller ions despite the flexibility of its backbone.

This conclusion is further supported by decomposing the relative complexation free energy of forming the La complex with ODA over Gd or Lu with respect to the reference solvated states. First, in agreement with extraction trends shown in FIG. 1B, the present experiments confirm that the relative binding free energy, ΔΔG_{bind} (solv) for La³⁺ is more favorable by 3.6 kcal/mol over Gd³⁺ and by 9.2 kcal/mol over Lu³⁺ (Table 1 below). To elucidate the origin of this stabilization for the larger La³⁺ ion, the present work partitioned the gas phase contribution of ΔΔE_{bind} (gas), which follows the same trend as the solution phase contribution, into the relative interaction energy between the ligand and the metal ion, ΔΔEᵢₙₜ, and the relative strain energy of the ligand, ΔΔEₛₜᵣ.

TABLE 1
Computed relative binding free energy in solvent (ΔΔG_{bind}(solv)) and
electronic binding energy in gas phase (ΔΔE_{bind}(gas)) for complexation
with ODA_{Me}. ΔE_{bind}(gas) is further decomposed into interaction
energy (ΔΔEᵢₙₜ) and strain energy (ΔΔEₛₜᵣ) in gas phase.

| M | Gd | Lu |
|---|---|---|
| ΔΔG_{bind} (solv) | −3.58 | −9.20 |
| ΔΔE_{bind} (gas) | −6.68 | −14.90 |
| ΔΔEᵢₙₜ (gas) | −9.36 | −15.87 |
| ΔΔEₛₜᵣ (gas) | 2.68 | 0.96 |

Values are provided in kcal/mol relative to La³⁺, which was set to 0.

It has herein been found that ΔΔEᵢₙₜ is a dominant contribution to ΔΔE_{bind} (gas), with ΔΔEₛₜᵣ playing a secondary role. While the present work confirms the conventional understanding that a smaller metal ion induces stronger strain on both the ODAMe ligand and the reference solvated Ln (H₂O) ₃ (NO₃)₃ system, the difference in the relative strain energy between the ligand and the reference system for a pair of metal ions is rather small. A positive value of ΔΔEₛₜᵣ indicates that the ODAMe ligand experiences less strain compared to the reference system for Gd³⁺ or Lu³⁺ over La³⁺. This trend would not be generally expected, except when the smaller metal ion fails to strongly interact with all the donor atoms in the ligand, as seen from the DFT calculations and AIMD simulations. As such, the balance between electronic stabilization and strain results in a situation where the thermodynamic selectivity of ODAMe for La³⁺ is driven by more favorable interaction energies and electronic stabilization throughout the entirety of the complex vs. on a per-bond basis.

### CONCLUSIONS

Combining separations, spectroscopic, and computational results, the present experiments gained insight into the interplay of bonding and dynamics in the reverse Ln³⁺ size selectivity of a new conformationally flexible ligand, ODA. It has herein been shown that the stability and extraction of Ln³⁺-ODA complexes depend on the ability of the flexible ligand backbone to wrap around a given Ln³⁺ to saturate its coordination sphere. For smaller, more charge dense Ln³⁺ ions, ODA can form some individually stronger bonds (evidenced by FTIR and DFT results) with the metal center, but due to limited complementarity, other bonding interactions are weakened relative to expectations. This frustrated coordination results in large conformational fluctuations (2D IR spectral diffusion, AIMD) and poor extraction selectivity at the macroscale. By contrast, individually weaker bonds are formed with the larger, less charge dense La³⁺ ion. However, due to conformational matching, ODA is better able to coordinate, resulting in complexes with small conformational fluctuations and high thermodynamic stability. This mechanistic framework derived from synthesis, separations, spectroscopy, and theory point to a new paradigm in which conformational flexibility can be incorporated into ligand design to provide unconventional chemical selectivity.

While there have been shown and described what are at present considered the preferred embodiments of the invention, those skilled in the art may make various changes and modifications which remain within the scope of the invention defined by the appended claims.

What is claimed is:

1. A metal chelator compound having the following structure: 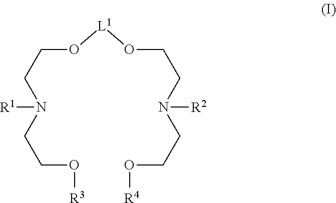
wherein:
L¹ is a linker containing 2-8 carbon atoms optionally containing one or more heteroatoms selected from O, N, and S atoms;
R¹ and R² are independently selected from hydrocarbon groups containing 1-12 carbon atoms optionally containing one or more heteroatoms selected from O, N, and S atoms, and each one of R¹ and R² is substituted with at least one heteroatom-containing group selected from the group consisting of: carboxy groups of the formula —C(O) OR′; phosphoryl groups of the formula —P(═O)(OH)R″; oxy groups of the formula OR′; amino groups of the formula —NR′₂; and amido groups of the formula —C(O)NHR′₂; wherein R′ is independently selected from H and hydrocarbon groups containing 1-6 carbon atoms and R″ is selected from OH and hydrocarbon groups containing 1-6 carbon atoms; and
R³ and R⁴ are independently selected from hydrocarbon groups containing 4-30 carbon atoms, wherein R³ and R⁴ are optionally partially or fully fluorinated and/or optionally contain a single O, S, NH, or N(CH₃) linkage interrupting a carbon-carbon bond.

2. The compound of claim 1, wherein the compound has any one of the following formulas: 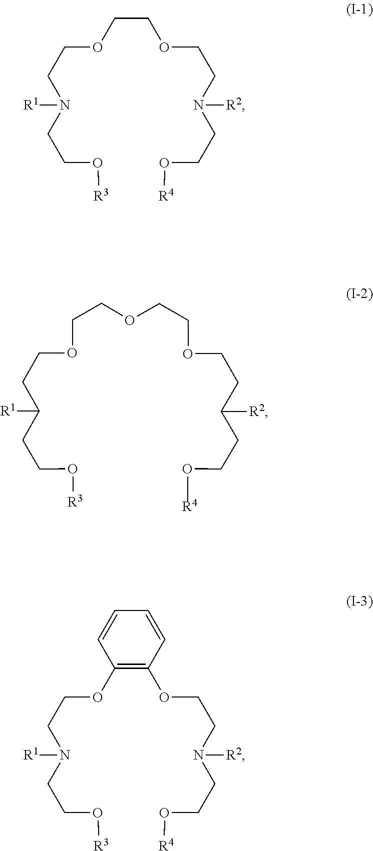
wherein R¹, R², R³, and R⁴ are as defined in claim 1.

3. The compound of claim 1, wherein R¹ and R² are independently selected from:

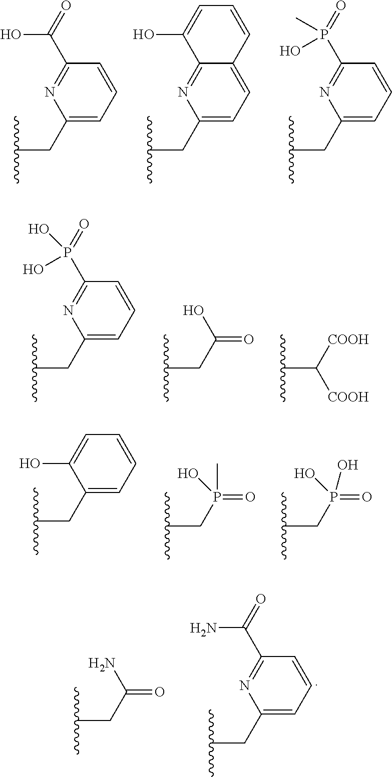

4. The compound of claim 1, wherein R³ and R⁴ are independently selected from hydrocarbon groups containing 5-30 carbon atoms.

5. The compound of claim 1, wherein R³ and R⁴ are independently selected from hydrocarbon groups containing 6-30 carbon atoms.

6. The compound of claim 1, wherein the compound of Formula (I) has the following structure: 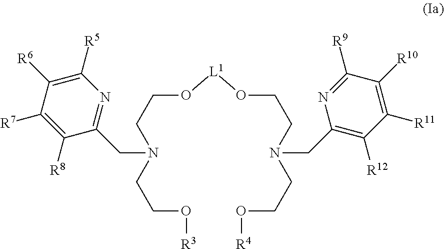
wherein:
L¹, R³, and R⁴ are as defined in claim 1;
R⁵, R⁶, R⁷, R⁸, R⁹, R¹⁰, R¹¹, and R¹² are independently selected from H, hydrocarbon groups containing 1-3 carbon atoms; carboxy groups of the formula —C(O) OR′; phosphoryl groups of the formula —P(═O)(OH)R″; oxy groups of the formula OR′; amino groups of the formula —NR′₂; and amido groups of the formula —C(O)NHR′₂; wherein R′ is independently selected from H and hydrocarbon groups containing 1-6 carbon atoms and R″ is selected from OH and hydrocarbon groups containing 1-6 carbon atoms;
wherein a pair of adjacent groups selected from R⁵, R⁶, R⁷, and R⁸ and/or a pair of adjacent groups selected from R⁹, R¹⁰, R¹¹, and R¹² are optionally interconnected to form an additional ring to form a fused ring system, wherein the additional ring may optionally and independently be substituted with one or more hydrocarbon groups containing 1-3 carbon atoms; carboxy groups of the formula —C(O) OR′; phosphoryl groups of the formula —P(═O)(OH)R″; oxy groups of the formula OR′; amino groups of the formula —NR′₂; and amido groups of the formula —C(O)NHR′₂, wherein R′ is independently selected from H and hydrocarbon groups containing 1-6 carbon atoms and R″ is selected from OH and hydrocarbon groups containing 1-6 carbon atoms; and
X¹ is selected from CR^{a} and N, wherein R^{a} is selected from hydrocarbon groups containing 1-3 linearly connected carbon atoms; carboxy groups of the formula —C(O) OR′; phosphoryl groups of the formula —P(═O)(OH)R″; oxy groups of the formula OR′; amino groups of the formula —NR′₂; and amido groups of the formula —C(O)NHR′₂; wherein R′ is independently selected from H and hydrocarbon groups containing 1-6 carbon atoms and R″ is selected from OH and hydrocarbon groups containing 1-6 carbon atoms;
X² is selected from CR^{b} and N, wherein R^{b} is selected from hydrocarbon groups containing 1-3 linearly connected carbon atoms; carboxy groups of the formula —C(O) OR′; phosphoryl groups of the formula —P(═O)(OH)R″; oxy groups of the formula OR′; amino groups of the formula —NR′₂; and amido groups of the formula —C(O)NHR′₂; wherein R′ is independently selected from H and hydrocarbon groups containing 1-6 carbon atoms and R″ is selected from OH and hydrocarbon groups containing 1-6 carbon atoms;
wherein at least one of R^{a}, R⁵, R⁶, R⁷, and R⁸ is selected from the group consisting of said carboxy, phosphoryl, oxy, amino, and amido groups, and at least one of R^{b}, R⁹, R¹⁰, R¹¹, and R¹² is selected from the group consisting of said carboxy, phosphoryl, oxy, amino, and amido groups.

7. The compound of claim 6, wherein the compound has any one of the following formulas:

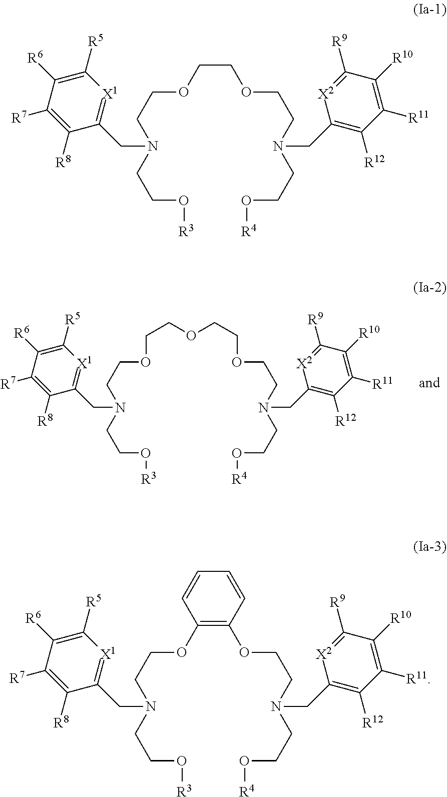

8. The compound of claim 6, wherein R³ and R⁴ are independently selected from hydrocarbon groups containing 5-30 carbon atoms.

9. The compound of claim 6, wherein R³ and R⁴ are independently selected from hydrocarbon groups containing 6-30 carbon atoms.

10. The compound of claim 6, wherein the compound of Formula (I) has the following structure:

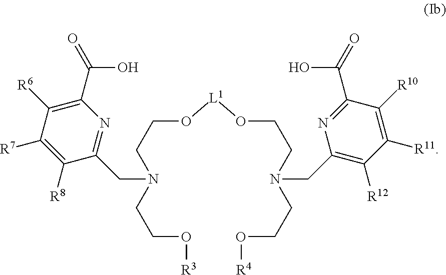

11. The compound of claim 10, wherein the compound has any one of the following formulas:

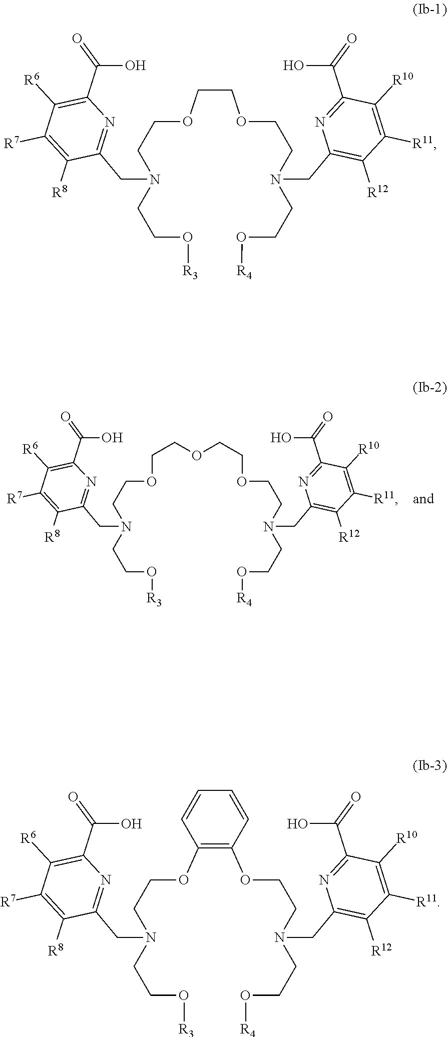

12. The compound of claim 10, wherein R³ and R⁴ are independently selected from hydrocarbon groups containing 5-30 carbon atoms.

13. The compound of claim 10, wherein R³ and R⁴ are independently selected from hydrocarbon groups containing 6-30 carbon atoms.

14. An aqueous-insoluble hydrophobic solution useful for extracting rare earth elements from aqueous solutions, the aqueous-insoluble hydrophobic solution comprising a rare earth extractant compound dissolved in an aqueous-insoluble hydrophobic solvent, wherein the rare earth extractant compound has the following structure: 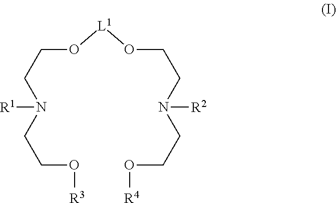
wherein:
L′ is a linker containing 2-8 carbon atoms optionally containing one or more heteroatoms selected from O, N, and S atoms;
R¹ and R² are independently selected from hydrocarbon groups containing 1-12 carbon atoms optionally containing one or more heteroatoms selected from O, N, and S atoms, and each one of R¹ and R² is substituted with at least one heteroatom-containing group selected from the group consisting of: carboxy groups of the formula —C(O) OR′; phosphoryl groups of the formula —P(═O)(OH)R″; oxy groups of the formula OR′; amino groups of the formula —NR′₂; and amido groups of the formula —C(O)NHR′₂; wherein R′ is independently selected from H and hydrocarbon groups containing 1-6 carbon atoms and R″ is selected from OH and hydrocarbon groups containing 1-6 carbon atoms; and
R³ and R⁴ are independently selected from hydrocarbon groups containing 4-30 carbon atoms.

15. The aqueous-insoluble hydrophobic solution of claim 14, wherein R³ and R⁴ are independently selected from hydrocarbon groups containing 5-30 carbon atoms.

16. The aqueous-insoluble hydrophobic solution of claim 14, wherein R³ and R⁴ are independently selected from hydrocarbon groups containing 6-30 carbon atoms.

17. The aqueous-insoluble hydrophobic solution of claim 14, wherein the aqueous-insoluble hydrophobic solvent is selected from a non-halogenated hydrocarbon solvent, halogenated hydrocarbon solvent, ether solvent, or alcohol solvent containing at least six carbon atoms.

18. A metal-ligand complex comprising the following components:
(i) a metal chelator compound of the following formula: 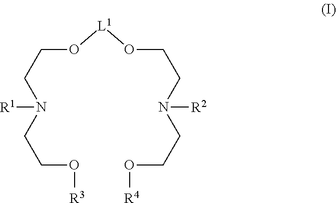
wherein:
L¹ is a linker containing 2-8 carbon atoms optionally containing one or more heteroatoms selected from O, N, and S atoms;
R¹ and R² are independently selected from hydrocarbon groups containing 1-12 carbon atoms optionally containing one or more heteroatoms selected from O, N, and S atoms, and each one of R¹ and R² is substituted with at least one heteroatom-containing group selected from the group consisting of: carboxy groups of the formula —C(O) OR′; phosphoryl groups of the formula —P(═O)(OH)R″; oxy groups of the formula OR′; amino groups of the formula —NR′₂; and amido groups of the formula —C(O)NHR′₂; wherein R′ is independently selected from H and hydrocarbon groups containing 1-6 carbon atoms and R″ is selected from OH and hydrocarbon groups containing 1-6 carbon atoms; and
R³ and R⁴ are independently selected from hydrocarbon groups containing 4-30 carbon atoms; and
(ii) a metal (M) complexed with said metal chelator compound.

19. The metal-ligand complex of claim 18, wherein R³ and R⁴ are independently selected from hydrocarbon groups containing 5-30 carbon atoms.

20. The metal-ligand complex of claim 18, wherein R³ and R⁴ are independently selected from hydrocarbon groups containing 6-30 carbon atoms.

21. The metal-ligand complex of claim 18, wherein said metal is a rare earth metal.

22. The metal-ligand complex of claim 21, wherein said rare earth metal is selected from the group consisting of lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), yttrium (Y), and scandium (Sc).

23. A method of at least partially separating rare earth (RE) elements from an aqueous solution containing at least two rare earth elements, the method comprising contacting said aqueous solution with an aqueous-insoluble hydrophobic solution comprising a rare earth extractant compound dissolved in an aqueous-insoluble hydrophobic solvent, wherein the rare earth extractant compound has the following structure: 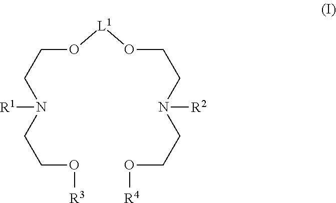
wherein:
L¹ is a linker containing 2-8 carbon atoms optionally containing one or more heteroatoms selected from O, N, and S atoms;
R¹ and R² are independently selected from hydrocarbon groups containing 1-12 carbon atoms optionally containing one or more heteroatoms selected from O, N, and S atoms, and each one of R¹ and R² is substituted with at least one heteroatom-containing group selected from the group consisting of: carboxy groups of the formula —C(O) OR′; phosphoryl groups of the formula —P(═O)(OH)R″; oxy groups of the formula OR′; amino groups of the formula —NR′₂; and amido groups of the formula —C(O)NHR′₂; wherein R′ is independently selected from H and hydrocarbon groups containing 1-6 carbon atoms and R″ is selected from OH and hydrocarbon groups containing 1-6 carbon atoms; and
R³ and R⁴ are independently selected from hydrocarbon groups containing 4-30 carbon atoms;
wherein, after contact, the aqueous-insoluble hydrophobic solution contains a higher molar ratio of lower atomic number or larger radii RE element(s) over higher atomic number or smaller radii RE element(s) compared to the molar ratio of lower atomic number or larger radii RE element(s) over higher atomic number or smaller radii RE element(s) in the aqueous solution.

24. The method of claim 23, wherein R³ and R⁴ are independently selected from hydrocarbon groups containing 5-30 carbon atoms.

25. The method of claim 23, wherein R³ and R⁴ are independently selected from hydrocarbon groups containing 6-30 carbon atoms.

26. The method of claim 23, wherein the aqueous-insoluble hydrophobic solvent is a hydrocarbon solvent.

27. The method of claim 23, wherein the aqueous solution further comprises a buffer that maintains the aqueous solution in a pH in a range of 2-7.

28. The method of claim 23, wherein the aqueous solution further comprises an electrolyte.

29. The method of claim 23, wherein said at least two rare earth elements are selected from the group consisting of lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), yttrium (Y), and scandium (Sc).

30. The method of claim 23, wherein the method achieves at least partial selective extraction into the aqueous-insoluble hydrophobic solution of one or more lighter lanthanide element(s) having an atomic number in a range of 57-65 over one or more heavier lanthanide element(s) having an atomic number in a range of 66-71, wherein the aqueous solution contains at least one element selected from said lighter lanthanide elements and at least one element selected from said heavier lanthanide elements.
